(19) United States
(12) Patent Application Publication
(10) Pub. No.: US 2025/0268555 A1
(43) Pub. Date: Aug. 28, 2025

(54) RADIATION DETECTION APPARATUS, RADIATION DETECTION SYSTEM, CONTROL APPARATUS, CONTROL METHOD, AND STORAGE MEDIUM

(71) Applicant: CANON KABUSHIKI KAISHA, Tokyo (JP)

(72) Inventors: TOMOYASU MIZUNO, Tokyo (JP); TOMOYA SUZUKI, Tokyo (JP)

(21) Appl. No.: 19/208,062

(22) Filed: May 14, 2025

Related U.S. Application Data
(63) Continuation of application No. PCT/JP2023/041491, filed on Nov. 17, 2023.

(30) Foreign Application Priority Data
Nov. 28, 2022 (JP) 2022-189348
Nov. 30, 2022 (JP) 2022-190923
Oct. 4, 2023 (JP) 2023-172594

(51) Int. Cl. A61B 6/00; A61B 6/42; A61B 6/58

(52) U.S. Cl. CPC A61B 6/547; A61B 6/4291; A61B 6/582

(57) ABSTRACT

A radiation detection system including a radiation source for irradiating radiation and a radiation detection apparatus for detecting the radiation includes an optical image capturing unit for obtaining an optical image by capturing an image of the radiation detection apparatus, and one or more controllers configured to obtain information about an orientation angle change and/or information about a position change output from a sensor unit included in the radiation detection apparatus, obtain information about a reference orientation angle and/or information about a reference position of the radiation detection apparatus based on the optical image, and obtain information about an orientation angle at a predetermined timing based on the information about the reference orientation angle and the information about the orientation angle change and/or information about a position at a predetermined timing based on the information about the reference position and the information about the position change.

### CROSS-REFERENCE TO RELATED APPLICATIONS

This application is a Continuation of International Patent Application No. PCT/JP2023/041491, filed Nov. 17, 2023, which claims the benefit of Japanese Patent Applications No. 2022-189348, filed Nov. 28, 2022, No. 2022-190923, filed Nov. 30, 2022, and No. 2023-172594, filed Oct. 4, 2023, all of which are hereby incorporated by reference herein in their entirety.

### BACKGROUND OF THE INVENTION

### Field of the Invention

The present disclosure relates to a radiation detection apparatus, a radiation detection system, a control apparatus, a control method, and a storage medium. This radiation detection apparatus can be applied to a dedicated apparatus for auto exposure control, a radiation imaging apparatus including an auto exposure mechanism, and the like.

### Background Art

A flat panel detector (FPD) formed of a semiconductor material serving as a radiation detector for medical diagnostic imaging or non-destructive inspection using radiation such as an X-ray has been in widespread use. A radiation imaging system using such a radiation detector in combination with a radiation generation apparatus or the like for generating radiation has been used.

It is known that, in such a radiation imaging system, radiation cannot reach homogenously the radiation imaging apparatus due to various factors such as a positional relationship between the radiation imaging apparatus and a radiation source.

PTL 1 discusses a technique for detecting the amount of radiation passing through direct incidence portions located at four corners of a radiation imaging apparatus and measuring a variation in the arrival amount of radiation, thereby correcting a radiological image in which the variation is taken into account. PTL 1 also discusses an auto exposure control (AEC) technique for stopping irradiation of radiation from a radiation source.

PTL 2 discusses a technique for deriving orientations of a radiation generation apparatus and a radiation imaging apparatus and displaying the orientations on a display unit or the like, thereby supporting the alignment between an irradiation field plane of radiation to be irradiated from the radiation generation apparatus and an incidence plane of the radiation imaging apparatus. According to the technique discussed in PTL 2, the radiation imaging apparatus is provided with an input unit for setting a reference orientation and an alignment rule serving as a structure for positioning. When the radiation imaging apparatus is brought into contact with the alignment rule, a user of the radiation imaging apparatus issues an instruction to set the reference orientation (calibration processing) via the input unit, thereby making it possible to set the reference orientation.

### CITATION LIST

### Patent Literature

PTL 1: Japanese Patent Application Laid-Open No. 2016-209457

PTL 2: Japanese Patent Application Laid-Open No. 2021-45647

In the case of using information about the positional relationship between the radiation generation apparatus and the radiation imaging apparatus to control radiation imaging as in the technique discussed in PTL 1, the positional relationship information is required to have an adequate accuracy. It may thus be desirable to take some measures by, for example, frequently setting the reference orientation as in the technique discussed in PTL 2. However, such a method may increase the time and labor for a user, such as an engineer, and thus is undesirable.

### SUMMARY OF THE INVENTION

The present disclosure is directed to providing a technique for accurately deriving an orientation of a radiation imaging apparatus while reducing the time and effort for a user, such as an engineer, to perform an operation.

The above-described issues can be solved by a radiation detection system including a radiation source configured to irradiate radiation and a radiation detection apparatus configured to detect the radiation, the radiation detection system including an optical image capturing unit configured to obtain an optical image by capturing an image of the radiation detection apparatus, and one or more controllers configured to obtain information about an orientation angle change and/or information about a position change output from a sensor unit included in the radiation detection apparatus, obtain information about a reference orientation angle and/or information about a reference position of the radiation detection apparatus based on the optical image, and obtain information about an orientation angle at a predetermined timing based on the information about the reference orientation angle and the information about the orientation angle change and/or information about a position at a predetermined timing based on the information about the reference position and the information about the position change.

Further features of the present invention will become apparent from the following description of exemplary embodiments with reference to the attached drawings.

### BRIEF DESCRIPTION OF THE DRAWINGS

FIG. 1 illustrates a configuration example of a radiation imaging system.

FIG. 2 illustrates a hardware configuration example of a radiation imaging apparatus.

FIG. 3 is a functional block diagram of the radiation imaging system.

FIG. 4 is a diagram illustrating a use sequence of the radiation imaging system.

FIG. 5 is a diagram illustrating a sequence for preprocessing by taking into account an orientation.

FIG. 6 illustrates an example of a radiation imaging management screen.

FIG. 7 illustrates an example of a setting screen for making settings by taking into account the orientation.

FIG. 8A illustrates an angular relationship between a radiation source and the radiation imaging apparatus.

FIG. 8B is a table for correction processing in image capturing with a facing orientation.

FIG. 8C is a table for correction processing in which an inclination is not taken into account in image capturing with an inclined orientation.

FIG. 8D is a table for correction processing in which an inclination is taken into account in image capturing with an inclined orientation.

FIG. 9 is a flowchart illustrating exposure control processing.

FIG. 10 is a flowchart illustrating image obtaining processing.

FIG. 11A illustrates a state of attenuation of radiation in a case where a grid is located between the radiation imaging apparatus and the radiation source that are directly opposite to each other.

FIG. 11B illustrates a state of attenuation of radiation in a case where a grid is located between the radiation imaging apparatus and the radiation source that are not directly opposite to each other.

FIG. 11C illustrates a state of attenuation of radiation due to a heel effect when the radiation source is located in a predetermined direction.

FIG. 11D illustrates a state of attenuation of radiation due to the heel effect when the radiation source is located in a different direction.

FIG. 11E illustrates a distribution of an arrival amount of radiation in a case where the radiation source faces the center of the radiation imaging apparatus.

FIG. 11F illustrates a distribution of the arrival amount of radiation in a case where the radiation source does not face the center of the radiation imaging apparatus.

FIG. 12 illustrates a radiation imaging system according to a second exemplary embodiment.

FIG. 13 illustrates a configuration example of the radiation imaging system according to the second exemplary embodiment.

FIG. 14 illustrates an example of markers on a radiation detection plane according to the second exemplary embodiment.

FIG. 15 illustrates a camera coordinate system according to the second exemplary embodiment.

FIG. 16 illustrates a camera image according to the second exemplary embodiment.

FIG. 17 illustrates a sensor coordinate system according to the second exemplary embodiment.

FIG. 18 illustrates a gravitational force coordinate system according to the second exemplary embodiment.

FIG. 19 is a flowchart illustrating calibration processing according to the second exemplary embodiment.

FIG. 20 illustrates an example of a home position according to a third exemplary embodiment.

FIG. 21 illustrates coordinates of each of a radiation source and a radiation imaging apparatus according to the third exemplary embodiment.

FIG. 22 is a flowchart illustrating calibration processing according to the third exemplary embodiment.

### DESCRIPTION OF THE EMBODIMENTS

Exemplary embodiments of the present invention will be described below with reference to the attached drawings. The detailed configurations described in the exemplary embodiments are not limited to those described in this specification and the drawings. Some or all of the configurations and processes described in the exemplary embodiments can be omitted or replaced within a range in which the effect of the present invention is obtained. For example, in this specification, X-rays are described as an example of radiation, but instead α-rays, β-rays, γ-rays, particle rays, cosmic rays, or the like can also be used as the radiation.

### First Exemplary Embodiment

### System Configuration

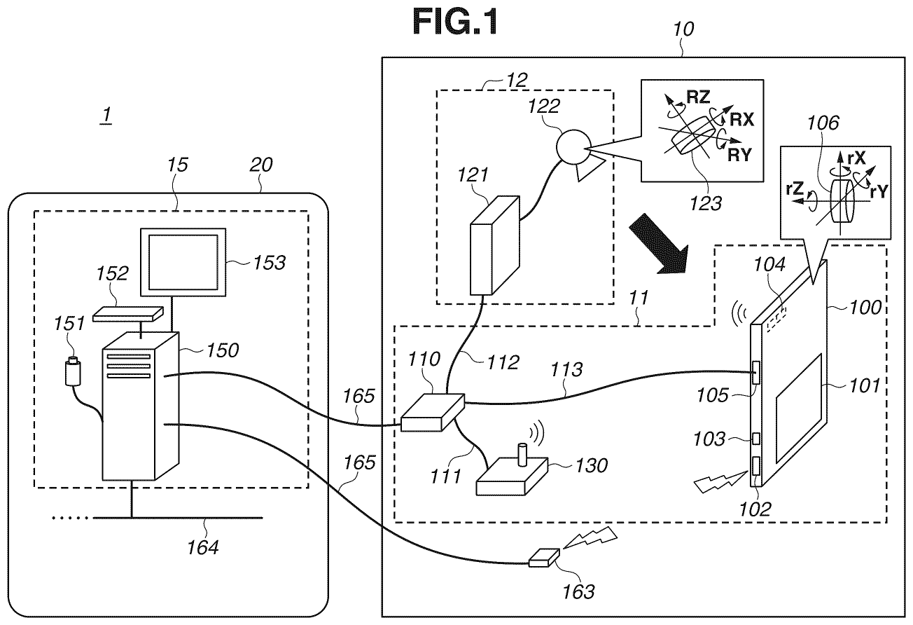

FIG. 1 illustrates a configuration example of a radiation imaging system.

As illustrated in FIG. 1, a radiation imaging system 1 (radiation detection system) includes a radiation room 10 for radiation imaging with radiation irradiation and a control room 20 located in the vicinity of the radiation room 10. As a configuration for implementing communication in each of the radiation room 10 and the control room 20, an in-hospital local area network (LAN) 164 and a radiation room communication cable 165, each of which is provided with an entry apparatus 163, are located.

In the radiation room 10, a radiation irradiation unit 12 and a radiation imaging unit 11 are located.

The radiation imaging unit 11 includes a radiation imaging apparatus 100, a communication control apparatus 110, an access point 130, an access point (AP) communication cable 111, a radiation generation apparatus communication cable 112, and a sensor communication cable 113.

The radiation imaging apparatus 100 (radiation image capturing apparatus) includes a power supply control unit 101 composed of a battery or the like, a near-field wireless communication unit 102, a switch 103, a wireless communication unit 104, a wired communication unit 105, and an angle sensor 106. The radiation imaging apparatus 100 detects radiation that is irradiated from a radiation source 122 of a radiation generation apparatus 121 and is transmitted through a subject (not illustrated), and generates radiological image data.

The access point 130 is an access point for establishing wireless communication and is used for the radiation imaging apparatus 100, the radiation generation apparatus 121, and an information processing apparatus 150 to communicate with each other via the communication control apparatus 110. The communication between the radiation imaging apparatus 100 and the communication control apparatus 110 can also be established by wired communication using the sensor communication cable 113. The present exemplary embodiment illustrates an example where the communication is established by wireless communication using the access point 130.

The radiation irradiation unit 12 includes the radiation generation apparatus 121 and the radiation source 122.

The radiation generation apparatus 121 controls the radiation source 122 and irradiates a subject with radiation (as indicated by an arrow in FIG. 1). The radiation generation apparatus 121 has a function for controlling the radiation irradiation using the radiation source 122, and a function for receiving a signal indicating start or stop of the irradiation from the radiation imaging apparatus 100. The radiation source 122 is a tube for radiation irradiation. The radiation source 122 includes an angle sensor 123 configured to detect an orientation.

The AP communication cable 111 is a cable for connecting the access point 130 and the communication control apparatus 110 to each other. The radiation generation apparatus communication cable 112 is a cable for connecting the radiation generation apparatus 121 and the communication control apparatus 110 to each other.

An information processing unit 15 is located in the control room 20. The information processing unit 15 includes the information processing apparatus 150, a radiation irradiation switch 151, an input apparatus 152, and a display apparatus 153.

The information processing apparatus 150 communicates with the radiation imaging apparatus 100 and the radiation generation apparatus 121 via the communication control apparatus 110, thereby controlling an overall operation of the radiation imaging system 1.

The radiation irradiation switch 151 is used for an operator (not illustrated) to input a radiation irradiation timing. The input apparatus 152 is an apparatus for inputting information from the operator. Various input devices such as a keyboard and a touch panel can be used. The display apparatus 153 is an apparatus configured to display radiological image data subjected to image processing and a graphical user interface (GUI). For example, a display is used as the display apparatus 153.

The in-hospital LAN 164 is a trunk network in a hospital. The radiation room communication cable 165 is a cable for connecting the information processing apparatus 150 in the control room 20 to the communication control apparatus 110 and the entry apparatus 163 in the radiation room 10.

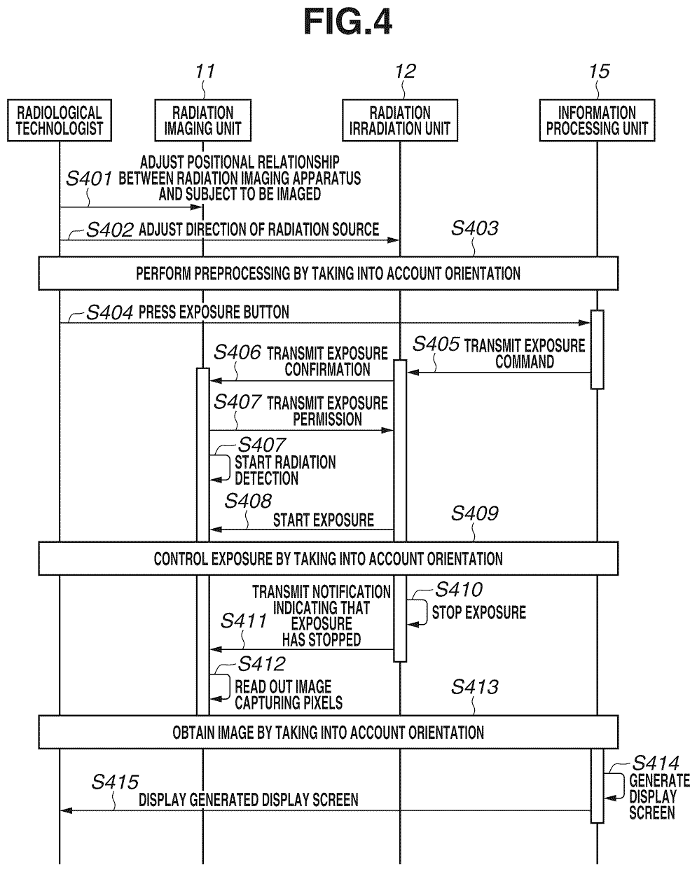

Next, a use sequence of the radiation imaging system 1 will be described. FIG. 4 illustrates a use sequence of the radiation imaging system 1.

To use the radiation imaging system 1, the operator takes various setting procedures in advance.

The operator performs a registration operation for the radiation imaging apparatus 100 to be used for radiation imaging. When the switch 103 of the radiation imaging apparatus 100 is pressed by the operator, near-field wireless communication is started between the near-field wireless communication unit 102 of the radiation imaging apparatus 100 and the entry apparatus 163.

The information processing apparatus 150 transmits wireless connection-related information about the access point 130 to the radiation imaging apparatus 100 via near-field wireless communication of the entry apparatus 163. In the case of using a wireless LAN, examples of the wireless connection-related information include communication methods, such as the Institute of Electrical and Electronics Engineers (IEEE) 802.11, a physical channel, a service set identifier (SSID), and an encryption key.

The radiation imaging apparatus 100 makes a setting for the wireless communication unit 104 based on received wireless LAN connection-related information. According to the setting, the radiation imaging apparatus 100 establishes a wireless communication connection with the access point 130.

Next, the operator inputs subject information, including an identification (ID), name, and date of birth of the subject, and an image capturing region of the subject, to the information processing apparatus 150. The operator inputs information about a dose, a maximum irradiation time, a tube current, a tube voltage, region information, and a radiation detection region (region of interest (ROI)), which is a region where radiation is to be monitored, to the information processing apparatus 150. As ROI information, a cumulative dose in each ROI and a threshold for a cumulative dose determination method can be input. Examples of the cumulative dose determination method in the case of using a plurality of ROIs include a logical AND when the cumulative doses in all ROIs to be monitored exceed the threshold, a logical OR when the cumulative dose in one or more ROI to be used exceeds the threshold, and an average value of cumulative doses in ROIs to be used exceeding the threshold.

If radiation is not monitored, there is no need to input ROI information. The information processing apparatus 150 transmits an input radiation irradiation condition, region information, ROI, and the like to the radiation imaging apparatus 100 and the radiation generation apparatus 121.

Thereafter, in step S401, a radiological technologist that is an operator adjusts a positional relationship between the radiation imaging apparatus 100 and a subject to be imaged.

In step S402, the radiological technologist adjusts the direction of the radiation source 122 such that the radiation source 122 is directed toward the center of the radiation imaging apparatus 100. To assist the direction adjustment, the radiation source 122 is provided with an illumination for illuminating in a radiation direction.

In step S403, preprocessing is performed by taking into account the orientation. This processing will be described in detail below.

In step S404, the operator presses the radiation irradiation switch 151 serving as an exposure switch after completion of preparation for imaging described above.

In step S405, the information processing unit 15 transmits an exposure command to the radiation irradiation unit 12.

In step S406, the radiation irradiation unit 12 transmits an exposure confirmation to the radiation imaging unit 11.

In step S407, the radiation imaging unit 11 transmits an exposure permission to the radiation irradiation unit 12. Along with this processing, radiation detection is started.

In step S408, the radiation irradiation unit 12 starts exposure. When the exposure is started, the radiation source 122 irradiates radiation to the subject. The radiation imaging apparatus 100 establishes wireless communication with the radiation generation apparatus 121, thereby controlling start and end of radiation irradiation. The radiation irradiated to the subject is transmitted through the subject and enters the radiation imaging apparatus 100. The radiation imaging apparatus 100 converts the incident radiation into visible light. The visible light is then detected as a radiological image signal by a photoelectric conversion element.

In step S409, an exposure control is performed by taking into account the orientation. This processing will be described in detail below.

In step S410, the radiation irradiation unit 12 performs exposure stop processing.

In step S411, a notification indicating that the exposure has stopped is transmitted to the radiation imaging unit 11.

In step S412, the radiation imaging unit 11 detects completion of radiation imaging, and reads out image capturing pixels. The radiation imaging apparatus 100 drives the photoelectric conversion element to read out the radiological image signal, and an analog-to-digital (AD) conversion circuit converts an analog signal into a digital signal, thereby obtaining radiological image data. The obtained radiological image data is transferred from the radiation imaging apparatus 100 to the information processing apparatus 150 by wireless communication.

In step S413, an image is obtained by taking into account the orientation. This processing will be described in detail below.

In step S414, the information processing unit 15 generates a display screen.

In step S415, the information processing unit 15 displays the display screen. The display screen is viewed by the operator.

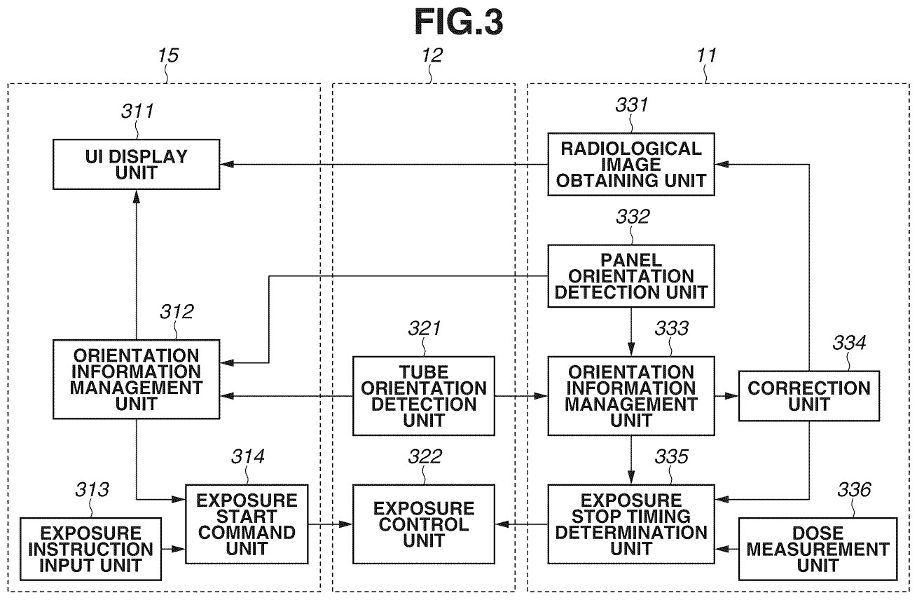

To implement the contents described in the sequence described above, it may be desirable for the radiation imaging system 1 to cooperate with various functions. FIG. 3 is a functional block diagram of the radiation imaging system 1. Processing to be executed by each functional block is implemented by any one of controllers (central processing units (CPUs)) in the radiation imaging system 1 executing a program. In the present exemplary embodiment, processing corresponding to functions of a user interface (UI) display unit 311, an orientation information management unit 312, an exposure instruction input unit 313, and an exposure start command unit 314 is executed in the information processing unit 15. In the radiation irradiation unit 12, processing corresponding to functions of a tube orientation detection unit 321 and an exposure control unit 322 is also executed. In the radiation imaging unit 11, processing corresponding to functions of a radiological image obtaining unit 331, a panel orientation detection unit 332, an orientation information management unit 333, a correction unit 334, an exposure stop timing determination unit 335, and a dose measurement unit 336 is also executed.

The panel orientation detection unit 332 is a functional block for detecting the orientation of the radiation imaging apparatus 100.

The tube orientation detection unit 321 is a functional block for detecting the orientation of the radiation source 122. The tube orientation detection unit 321 obtains orientation information from the angle sensor 123 included in the radiation source 122.

The orientation information management unit 312 is a functional block for obtaining orientation information from each of the tube orientation detection unit 321 and the panel orientation detection unit 332 and using the obtained orientation information for processing in the information processing unit 15.

The information managed by the orientation information management unit 312 is utilized for the UI display unit 311 and the exposure start command unit 314.

The exposure start command unit 314 is a functional block for controlling an exposure start command by taking into account the orientation information. Specifically, the exposure start command unit 314 transmits an exposure command to the exposure control unit 322 in a case where an input from the exposure instruction input unit 313 and orientation information input from the orientation information management unit 312 satisfy a specific condition.

The exposure instruction input unit 313 is a functional block for detecting pressing of the exposure switch by the operator.

The orientation information management unit 333 is a functional block for obtaining orientation information from each of the tube orientation detection unit 321 and the panel orientation detection unit 332 and using the obtained orientation information for processing in the radiation imaging unit 11. The orientation information management unit 333 transmits the orientation information to the correction unit 334.

The correction unit 334 is a functional block for performing processing of generating various correction information and processing of changing setting values based on the orientation information. Information related to an exposure stop timing in the correction information is transmitted to the exposure stop timing determination unit 335. Information related to radiological images in the correction information is transmitted to the radiological image obtaining unit 331.

The exposure stop timing determination unit 335 transmits an exposure stop command by taking into account the orientation information. Specifically, the exposure stop timing determination unit 335 transmits the exposure stop command to the exposure control unit 322 (communication processing is executed) in a case where an output from the dose measurement unit 336 and correction information from the correction unit 334 satisfy a specific condition,.

The exposure control unit 322 is a functional unit for performing control processing to start the exposure upon reception of the exposure start command, and performing control processing to stop the exposure upon reception of the exposure stop command.

The radiological image obtaining unit 331 is a functional block for generating a corrected radiological image based on an output of a captured image and correction information from the correction unit 334.

The radiological image obtaining unit 331 obtains correction data in which a correction amount for each pixel of radiological image data generated by the radiation imaging apparatus 100 is defined, from the correction unit 334. The correction data is managed together with the orientation of the radiation imaging apparatus 100 with respect to the radiation source 122 when the correction data is generated. When the correction data corresponding to the orientation information is obtained, the radiological image data is corrected pixel by pixel. The corrected functional block is transmitted to the UI display unit 311.

The UI display unit 311 is a functional block for displaying information in the system in which orientation information is taken into account. Upon reception of the orientation information from the orientation information management unit 312, the UI display unit 311 displays a screen including information about the orientation information. When the UI display unit 311 obtains a corrected radiological image, the UI display unit 311 displays the corrected radiological image.

### Hardware Configuration

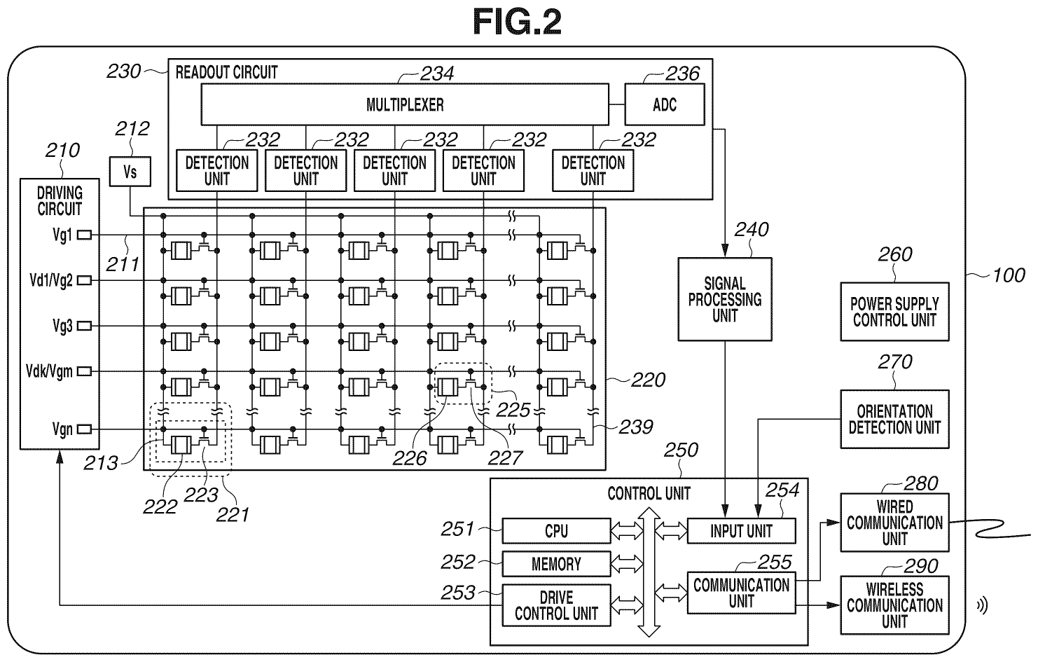

FIG. 2 illustrates a hardware configuration example of the radiation imaging apparatus 100.

As illustrated in FIG. 2, the radiation imaging apparatus 100 includes a radiation detector 220. The radiation detector 220 has a function for detecting irradiated radiation. The radiation detector 220 includes a plurality of pixels arrayed to form a plurality of rows and a plurality of columns. A region in which the plurality of pixels is located in the radiation detector 220 is hereinafter referred to as an image capturing region.

The plurality of pixels includes a plurality of image capturing pixels 221 for obtaining radiological image data, and detection pixels 225 for monitoring irradiation of radiation. The detection pixels 225 are pixels to be used for auto exposure control (auto exposure amount control).

Each image capturing pixel 221 includes a first conversion element 222 for converting radiation into an electrical signal, and a first switch 223 located between a column signal line 239 and the first conversion element 222.

Each detection pixel 225 includes a second conversion element 226 for converting radiation into an electrical signal, and a second switch 227 located between the column signal line 239 and the second conversion element 226. The detection pixel 225 is located in the same column as that of some of the plurality of image capturing pixels 221.

The first conversion element 222 and the second conversion element 226 are each composed of a scintillator that converts radiation into light and a photoelectric conversion element that converts light into an electrical signal. The scintillator is typically formed in a sheet shape so as to cover the image capturing region, and is shared by a plurality of pixels. Alternatively, the first conversion element 222 and the second conversion element 226 are each composed of a conversion element that directly converts radiation into an electrical signal.

The first switch 223 and the second switch 227 each include a thin film transistor (TFT) having a configuration in which an active region is formed of semiconductor, such as amorphous silicon or polycrystalline silicon (preferably, polycrystalline silicon).

The radiation imaging apparatus 100 includes a plurality of column signal lines 239 and a plurality of drive lines 211. Each column signal line 239 corresponds to one of the plurality of columns in the image capturing region. Each drive line 211 corresponds to one of the plurality of rows in the image capturing region. Each drive line 211 is driven by a driving circuit 210.

A first electrode of the first conversion element 222 is connected to a first main electrode of the first switch 223, and a second electrode of the first conversion element 222 is connected to a bias line 213. Each bias line 213 extends in a column direction and is commonly connected to the second electrodes of a plurality of first conversion elements 222 arrayed in the column direction.

Each bias line 213 receives a bias voltage Vs from an element power supply circuit 212. The bias voltage Vs is supplied from the element power supply circuit 212. A power supply control unit 260 is a unit corresponding to the power supply control unit 101 and is composed of a battery, a direct current (DC)-DC converter, or the like. The power supply control unit 260 includes the element power supply circuit 212 and generates power for analog circuits and power for digital circuits to perform drive control, wireless communication, and the like.

A second main electrode of the first switch 223 of each of the plurality of image capturing pixels 221 forming one column is connected to one column signal line 239. A control electrode of the first switch 223 of each of the plurality of image capturing pixels 221 forming one row is connected to one drive line 211. The plurality of column signal lines 239 is connected to a readout circuit 230. In this case, the readout circuit 230 includes a plurality of detection units 232, a multiplexer 234, and an analog-to-digital converter (ADC) 236 (AD converter, etc.).

Each of the plurality of column signal lines 239 is connected to a corresponding one of the plurality of detection units 232 of the readout circuit 230. One column signal line 239 corresponds to one detection unit 232. For example, each detection unit 232 includes a differential amplifier. The multiplexer 234 selects the plurality of detection units 232 in a predetermined sequence, and supplies signals from the selected detection units 232 to the AD converter 236. The AD converter 236 converts the supplied signals into digital signals, and outputs the digital signals. The output from the readout circuit 230 (AD converter 236) is supplied to a signal processing unit 240 and is processed by the signal processing unit 240. The signal processing unit 240 outputs information indicating irradiation of radiation to the radiation imaging apparatus 100 based on the output from the readout circuit 230 (AD converter 236).

A connection configuration of the second conversion element 226 of each detection pixel 225 is similar to a connection configuration of each image capturing pixel 221. In the case of driving the detection pixels 225, the driving circuit 210 drives the detection pixels 225 on the respective drive lines 211. When the detection pixels 225 are driven, the signal processing unit 240 outputs information indicating irradiation of radiation with respect to the radiation imaging apparatus 100 based on the output from the readout circuit 230 (AD converter 236). Specifically, the signal processing unit 240 detects, for example, irradiation of radiation to the radiation imaging apparatus 100 and calculates an irradiation amount and/or a cumulative irradiation amount of radiation.

Each detection pixel 225 can have the same structure as that of each image capturing pixel 221.

A control unit 250 controls the driving circuit 210, the readout circuit 230, and the like based on information from the signal processing unit 240 and a control command from the information processing apparatus 150.

The control unit 250 includes a CPU 251, a memory 252, a drive control unit 253, an input unit 254, and a communication unit 255.

The CPU 251 executes various programs stored in a storage medium of a program storage unit using the memory 252 as a working memory, thereby controlling an overall operation of the radiation imaging apparatus 100.

The memory 252 is a memory for storing various data to be handled by the CPU 251 and for reading and writing data. In the present exemplary embodiment, a random access memory (RAM) serving as a working memory and a flash read-only memory (ROM) serving as a storage unit are collectively referred to as the memory 252. However, the memory 252 can also be configured as a plurality of memories having different characteristics.

The drive control unit 253 controlled by the CPU 251 controls the driving circuit 210, the readout circuit 230, and the like based on information from the signal processing unit 240 and a command from the information processing apparatus 150. The communication unit 255 controlled by the CPU 251 communicates with the radiation generation apparatus 121 and the information processing apparatus 150 via the communication control apparatus 110 using a wireless communication unit 290 (corresponding to the wireless communication unit 104) or a wired communication unit 280 (corresponding to the wired communication unit 105).

The input unit 254 receives an input of information from the signal processing unit 240, and receives an input of information from an orientation detection unit 270. The orientation detection unit 270 is an angle sensor configured to obtain orientation information about the radiation imaging apparatus 100. The orientation detection unit 270 corresponds to the angle sensor 106 illustrated in FIG. 1. The orientation detection unit 270 is configured to detect angular information about an X-rotation direction, a Y-rotation direction, and a Z-rotation direction. The orientation detection unit 270 is a sensor that is used with an acceleration sensor, an angular velocity sensor, and a magnetic sensor singly or in combination. Assume that the angle sensor 123 also has the same function as the orientation detection unit 270.

### Preprocessing Taking Into Account Orientation

The display apparatus 153 displays various information to assist a radiation imaging operation of the operator. In the present exemplary embodiment, a controller of the information processing apparatus 150 functioning as the UI display unit 311 displays information by taking into account the orientation information in step S403.

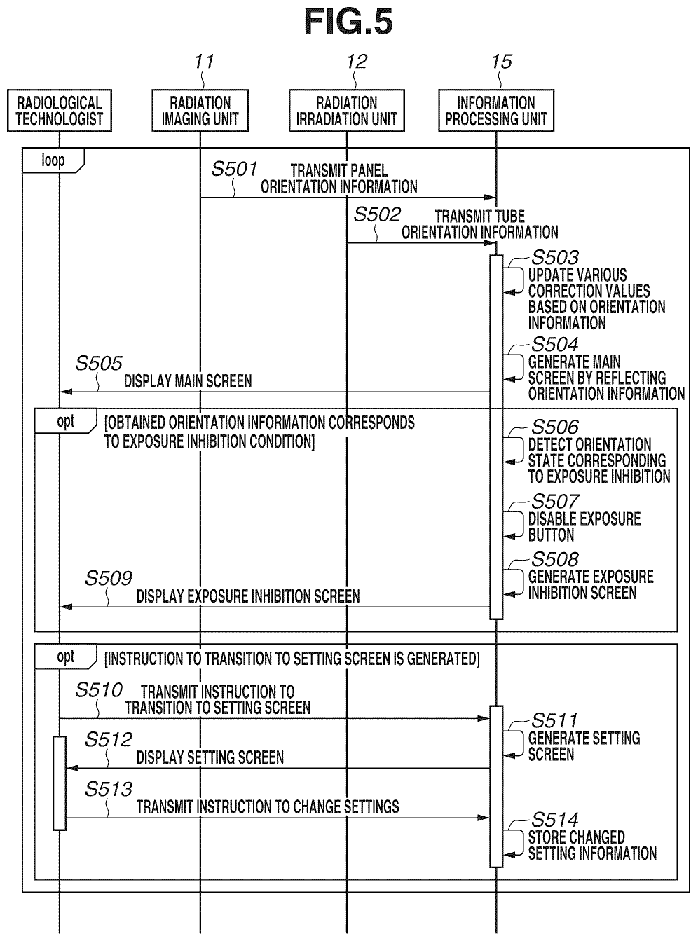

The processing of step S403 will be described in detail with reference to FIG. 5. FIG. 5 illustrates a sequence for preprocessing by taking into account the orientation.

In step S501, the radiation imaging unit 11 transmits panel orientation information to the information processing unit 15.

In step S502, the radiation irradiation unit 12 transmits tube orientation information to the information processing unit 15.

In step S503, the information processing unit 15 performs processing of updating various parameters influenced by the orientation information in radiation imaging.

In step S504, the information processing unit 15 generates a main screen reflecting the orientation information.

In step S505, the information processing unit 15 displays the main screen on the display apparatus 153 so that the radiological technologist can view the main screen.

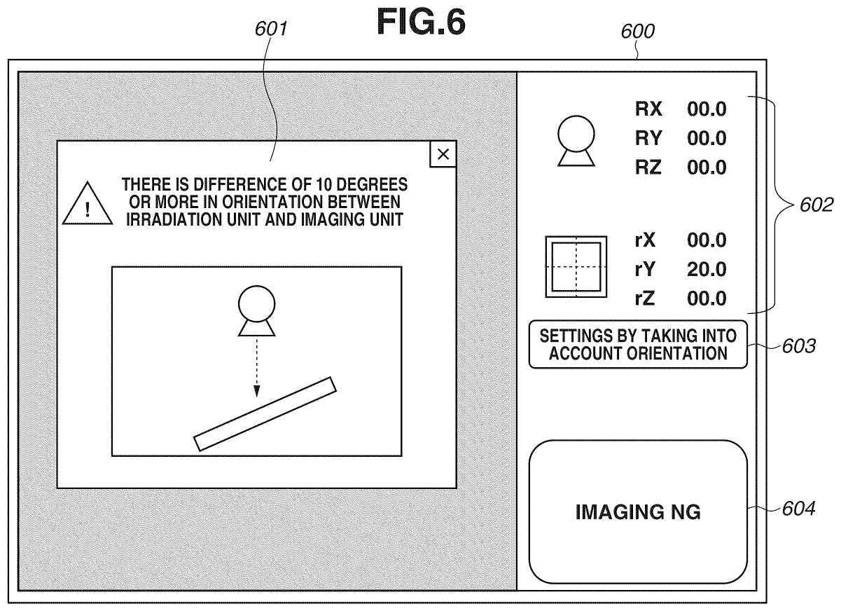

The main screen will be described with reference to FIG. 6. A main screen 600 includes information such as orientation information 602, a button 603, and status information 604. At this timing, a screen that does not include a message 601 is displayed (not illustrated).

The orientation information 602 is information indicating the orientation of the radiation source 122 and the orientation of the radiation imaging apparatus 100. The processing of step S403 is repeatedly performed, and thus the orientation information 602 is continuously updated in real time.

The button 603 is a button for causing the screen to transition to a setting screen for making settings by taking into account the orientation.

The status information 604 is an information unit to display information indicating whether exposure can be started.

During display of the main screen 600, processing of steps S506 to S509 is performed in a case where the obtained orientation information corresponds to an exposure inhibition condition.

In step S506, the information processing unit 15 detects an exposure inhibition orientation state. The term “exposure inhibition orientation state” used herein refers to a state where an angular difference between the radiation source 122 and the radiation imaging apparatus 100 is a predetermined angle or more from an ideal state. To detect the angular difference between the radiation source 122 and the radiation imaging apparatus 100, an angle in the X-rotation direction, an angle in the Y-rotation direction, or a combination of the angle in the X-rotation direction and the angle in the Y-rotation direction is used. In this operation, an angle in the Z-rotation direction can either be used or not be used.

In step S507, the information processing unit 15 disables an exposure start instruction issued by the radiation irradiation switch 151 serving as the exposure button, thereby inhibiting exposure start (irradiation start).

In step S508, the information processing unit 15 generates an exposure inhibition screen.

In step S509, the information processing unit 15 displays the exposure inhibition screen. The exposure inhibition screen is a screen obtained by superimposing the message 601 on the screen displayed in step S505. The message 601 is a pop-up window to inform that the orientation of the radiation source 122 and the orientation of the radiation imaging apparatus 100 are not preferable for radiation. On this pop-up window, a model for illustrating an angle relationship (orientation relationship) can also be located as well as a warning. The exposure inhibition screen displays status information 604 indicating “status NG”.

In a case where an instruction to transition to the setting screen is generated while the main screen 600 is displayed, processing of steps S510 to S514 is performed.

In step S510, the radiological technologist presses the button 603 to issue an instruction to transition to the setting screen.

In step S511, the information processing unit 15 generates the setting screen.

In step S512, the information processing unit 15 displays the setting screen on the display apparatus 153 so that the radiological technologist can view the setting screen.

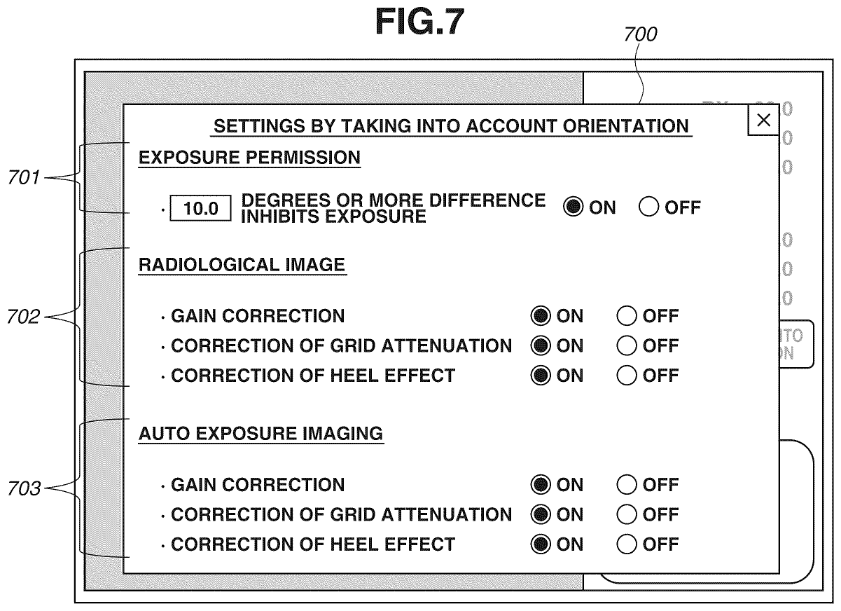

The setting screen will now be described with reference to FIG. 7. FIG. 7 illustrates an example of the setting screen for making settings by taking into account the orientation.

As illustrated in FIG. 7, a setting screen 700 includes exposure permission setting information 701, radiological image setting information 702, and auto exposure imaging setting information 703.

The exposure permission setting information 701 is configured to make an ON/OFF setting for inhibiting exposure based on the orientation, and to set (designate) a threshold for an angle at which the exposure is inhibited.

In the radiological image setting information 702, ON/OFF for various corrections on a radiological image can be set. Specifically, ON/OFF for gain correction, ON/OFF for grid attenuation correction, and ON/OFF for correction of a heel effect can be set.

In the auto exposure imaging setting information 703, ON/OFF for various corrections on an output from each dose detection pixel for auto exposure imaging can be set. Specifically, ON/OFF for gain correction, ON/OFF for grid attenuation correction, and ON/OFF for correction of the heel effect can be set.

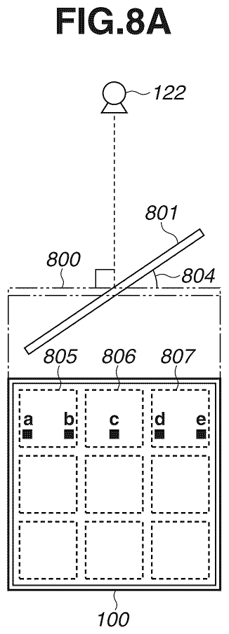

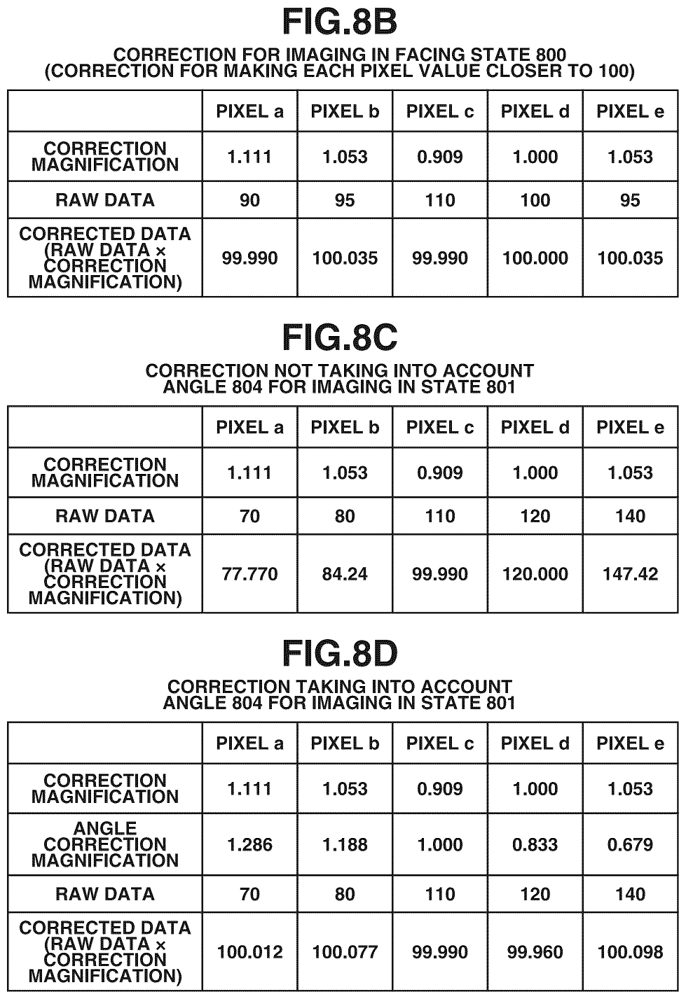

The gain correction according to the orientation will be described with reference to FIGS. 8A, 8B, 8C, and 8D. FIG. 8A illustrates an angular relationship between the radiation source 122 and the radiation imaging apparatus 100. FIG. 8B illustrates correction processing for image capturing with a facing orientation. FIG. 8C illustrates correction processing in which an inclination is not taken into account during image capturing with an inclined orientation. FIG. 8D illustrates correction processing in which an inclination is taken into account during image capturing with an inclined orientation.

As illustrated in FIG. 8A, the radiation imaging apparatus 100 can take a state 800 in which the radiation imaging apparatus 100 is directly opposite the radiation source 122, and a state 801 having an angular difference of an angle 804 without being directly opposite the radiation source 122. To show that the amount of radiation that reaches each pixel in the radiation imaging apparatus 100 from the radiation source 122 in the state 800 is different from that in the state 801, for example, representative pixels “a” to “e” among the plurality of pixels constituting the radiation detector 220 will be used in the following description. FIG. 8A illustrates a layout of the pixels “a” to “e” when the radiation imaging apparatus 100 is overlooked.

As indicated in a “raw data” row in FIG. 8B, the amount of radiation that reaches from the radiation source 122 tends to decrease toward an end from a maximum amount corresponding to the pixel “c” located at the center. It is generally known that radiation attenuates inversely with the square of a distance. In other words, the radiation amount gradually attenuates in a direction apart from the radiation source 122, and gradually increases in a direction approaching the radiation source 122. A variation in the radiation amount depending on the distance from the radiation source 122 in such a facing state is corrected as indicated by values in a “corrected data” row using values indicated in a “correction magnification” row by normal gain correction. This correction is performed on each pixel in the same manner, regardless of whether the pixels “a” to “e” correspond to the image capturing pixels 221 or the detection pixels 225. In FIG. 8A, a dashed line on the radiation imaging apparatus 100 indicates a ROI. While FIG. 8A illustrates an example where nine ROIs are set, any number of ROIs can be set and the layout of ROIs can be changed as appropriate. In FIG. 8A, the pixel “a” and the pixel “b” belong to a ROI 805, the pixel “c” belongs to a ROI 806, and the pixel “d” and the pixel “e” belong to a ROI 807. As seen from the tendency of “raw data” illustrated in FIG. 8B, an average dose value (cumulative dose value) of the detection pixel 225 included in the ROI 806 tends to be greater than an average dose value (cumulative dose value) in the ROI 805 and the ROI 807. Thus, when there is a variation in the average dose value (cumulative dose value) in each ROI, it is assumed that the timing of arrival at the threshold in each ROI varies. In other words, there is a possibility that an exposure stop notification timing can be delayed or accelerated depending on how to select the ROI to be used for auto exposure control. It is thus desirable to appropriately correct the dose value detected in each detection pixel 225.

Since the above-described “correction magnification” indicates a correction value assuming that the radiation source 122 and the radiation imaging apparatus 100 are directly opposite to each other, it is not appropriate to use the correction magnification in a state where the radiation source 122 and the radiation imaging apparatus 100 are not directly opposite to each other.

As illustrated in FIG. 8C, even when the “correction magnification” similar to that illustrated in FIG. 8B is applied to the “raw data” obtained in a state where the radiation source 122 and the radiation imaging apparatus 100 are not directly opposite to each other, the variation in the value cannot be appropriately suppressed as indicated by the “corrected data” row.

According to the present exemplary embodiment, the correction magnification corresponding to the angle 804 is further used as indicated by an “angle correction magnification” row illustrated in FIG. 8D, thereby suppressing the variation in the value as indicated in the “corrected data” row.

The above-described “correction magnification” is preliminarily determined experimentally or by artificial intelligence (AI) learning on the attenuation amount or increased amount of radiation, and a look-up table in which the correction magnification is managed is used. The attenuation amount or increased amount of radiation is determined using the look-up table depending on the orientation of the radiation source 122 and the orientation of the radiation imaging apparatus 100, thereby making it possible to appropriately perform correction processing. To obtain the correction magnification, a formula for calculating the attenuation amount or increased amount of radiation can be defined in advance and the correction magnification can be calculated each time.

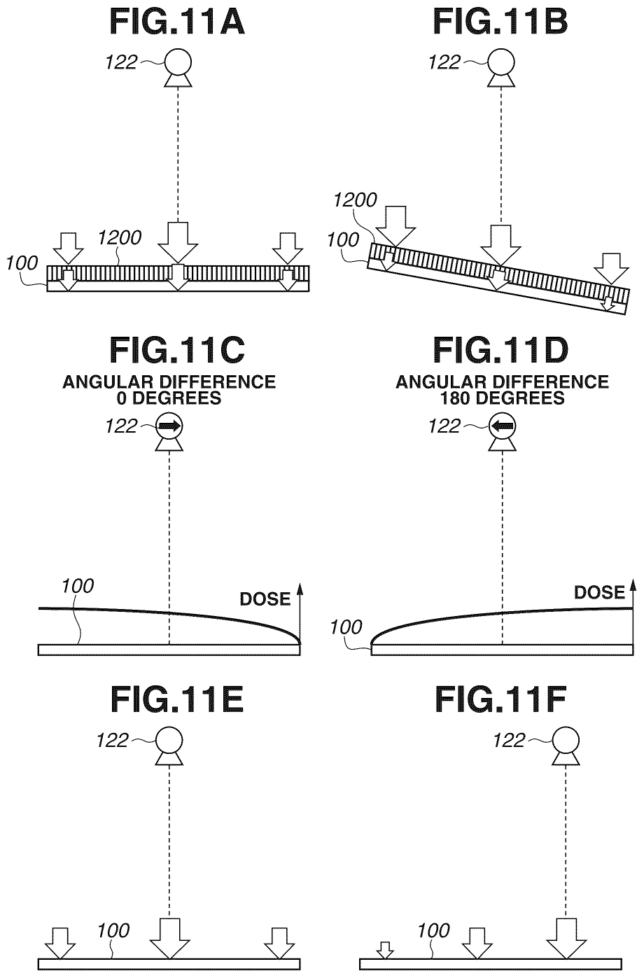

The grid attenuation correction according to the orientation will be described with reference to FIGS. 11A and 11B. FIG. 11A illustrates a state of attenuation of radiation in a case where a grid is located between the radiation source 122 and the radiation imaging apparatus 100 both of which are directly opposite to each other. FIG. 11B illustrates a state of attenuation of radiation in a case where a grid is located between the radiation source 122 and the radiation imaging apparatus 100 both of which are not directly opposite to each other.

A grid 1200, which is located to eliminate scattered rays, has an effect of attenuating the amount of radiation that reaches the radiation imaging apparatus 100. The amount of this attenuation varies depending on a slit in the grid 1200 and an angle formed by the radiation source 122. The attenuation amount of radiation at each position in the state where the radiation source 122 and the radiation imaging apparatus 100 are directly opposite to each other is thereby different from that in the state where the radiation source 122 and the radiation imaging apparatus 100 are not directly opposite to each other. According to the present exemplary embodiment, it is possible to perform correction processing of varying parameters to be used for grid attenuation correction depending on the angle 804 in the same manner as described above in FIGS. 8A and 8B.

The grid attenuation amount is influenced by the direction and pitch size of the grid. It can thus be desirable for the operator to input information about the direction (Z-rotation direction) or pitch size of the grid on the setting screen (not illustrated) in step S403.

The correction of the heel effect according to the orientation will be described with reference to FIGS. 11C and 11D. FIG. 11C illustrates a state of attenuation of radiation due to the heel effect in a case where the radiation source 122 is located in a predetermined direction. FIG. 11D illustrates a state of attenuation of radiation due to the heel effect in a case where the radiation source 122 is located in a different direction.

It is known that radiation from the radiation source 122 attenuates by an effect called the heel effect due to the configuration of the radiation source 122. How the heel effect influences varies depending on the direction in which an anode and a cathode are located in the radiation source 122. According to the present exemplary embodiment, it is possible to perform correction processing depending on the angular difference (Z-rotation direction) between the radiation source 122 and the radiation imaging apparatus 100.

In step S513, the radiological technologist operates the setting screen 700 to make a desired setting change.

In step S514, the information processing unit 15 reflects and holds the changed setting information.

As described above, in the present exemplary embodiment, a screen based on the orientation information is displayed, so that radiation imaging can be appropriately performed.

### Exposure Control Processing Taking Into Account Orientation

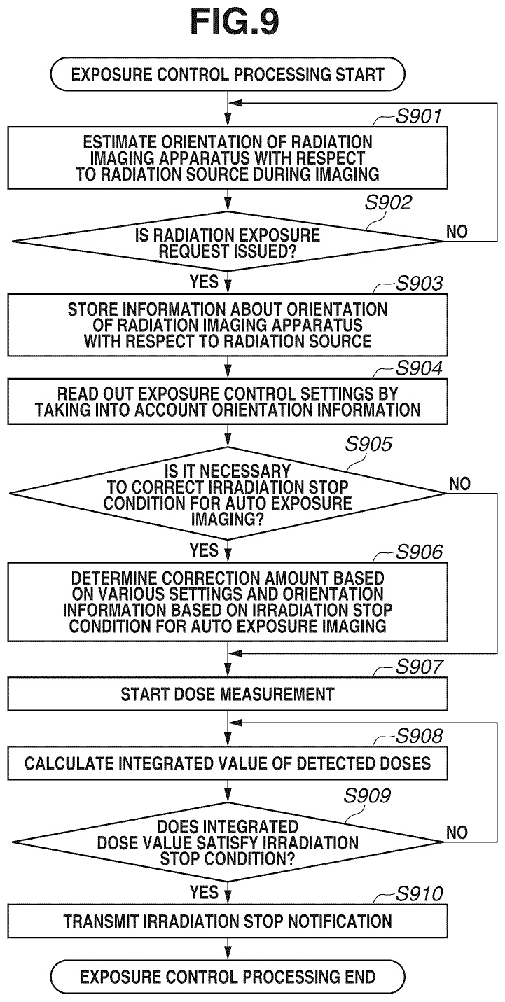

FIG. 9 is a flowchart illustrating exposure control processing. Each processing illustrated in this flowchart is implemented by the control unit 250 (in particular, the CPU 251) functioning as the corresponding functional unit illustrated in FIG. 3. Steps S901 and S902 are processing to be performed along with step S406. Steps S903 to S907 are processing to be performed along with step S407. Steps S908 to S910 are processing to be performed during step S409.

In step S901, the orientation information management unit 333 estimates the orientation of the radiation imaging apparatus 100 with respect to the radiation source 122 of the radiation generation apparatus 121 using the orientation information obtained from the tube orientation detection unit 321 and the panel orientation detection unit 332.

In step S902, the exposure instruction input unit 313 determines whether the radiation irradiation switch 151 is pressed and an exposure request is issued. In a case where it is determined that the exposure request is issued (YES in step S902), the processing proceeds to step S903. In a case where it is determined that the exposure request is not issued (NO in step S902), the processing returns to step S901.

In step S903, the orientation information management unit 333 holds the currently obtained orientation information.

In step S905, the orientation information management unit 333 determines whether it is necessary to correct the irradiation stop condition for auto exposure imaging (radiation imaging using auto exposure control). Whether it is necessary to correct the irradiation stop condition is determined based on the presence or absence of auto exposure control settings by taking into account the orientation, and based on the orientation state held in step S903. For example, it is determined that it is necessary to correct the irradiation stop condition in a case where any one of the auto exposure control settings by taking into account the orientation is ON and there is an angular difference of a predetermined value or more between the orientation of the radiation source 122 and the orientation of the radiation imaging apparatus 100.

In a case where the control unit 250 determines that it is necessary to correct the irradiation stop condition (YES in step S905), the processing proceeds to step S906. In a case where the control unit 250 determines that it is not necessary to correct the irradiation stop condition (NO in step S905), the processing proceeds to step S907.

In step S906, the correction unit 334 determines the correction amount based on the setting content in the setting information 703 and the orientation information, and transmits a notification about the correction amount to the exposure stop timing determination unit 335.

In step S907, the dose measurement unit 336 starts dose measurement.

In step S908, the exposure stop timing determination unit 335 detects radiation incident on the ROI set as a region of interest and corrects the detected radiation amount (dose value) with the correction value, and calculates a cumulative dose serving as an integrated value (cumulative value) of values obtained by performing correction processing.

In step S909, the exposure stop timing determination unit 335 refers to the cumulative dose (detection status) calculated in step S908 and determines whether the cumulative dose has reached a threshold (predetermined condition, predetermined value) that is a radiation irradiation stop condition. In a case where it is determined that the cumulative dose satisfies the irradiation stop condition (YES in step S909), the processing proceeds to step S910. In a case where it is determined that the cumulative dose does not satisfy the irradiation stop condition (NO in step S909), the processing returns to step S908.

In step S910, the exposure stop timing determination unit 335 transmits an irradiation stop notification to the exposure control unit 322 of the radiation generation apparatus 121 via the communication unit 255. The exposure control unit 322 stops irradiation of radiation based on the notified radiation irradiation stop timing. The radiation imaging apparatus 100 transmits the notification about the radiation irradiation stop timing as a result of detecting the radiation. However, the notification is not limited to this example. The radiation imaging apparatus 100 can transmit a cumulative dose every predetermined period as a detection result and the radiation generation apparatus 121 can calculate the integrated value of the cumulative doses. In this case, the correction value can be reflected at any timing. Before the radiation stop notification comes, the exposure control unit 322 can stop irradiation in a case where a preliminarily set maximum irradiation period has expired.

As described above, the radiation imaging system 1 according to the present exemplary embodiment estimates the distance attenuation of the radiation amount depending on the angle of the radiation source 122 with respect to the radiation imaging apparatus 100, the attenuation due to a grid, and the attenuation due to the heel effect, and obtains a correction value for calibration. Further, it is possible to prevent shortage of a dose in correction of an output from each dose detection pixel and in radiological image data captured for auto exposure control. Furthermore, it is possible to prevent an increase in the amount of radiation exposure of the subject due to a need for performing image capturing again.

### Image Obtaining Processing Taking Into Account Orientation

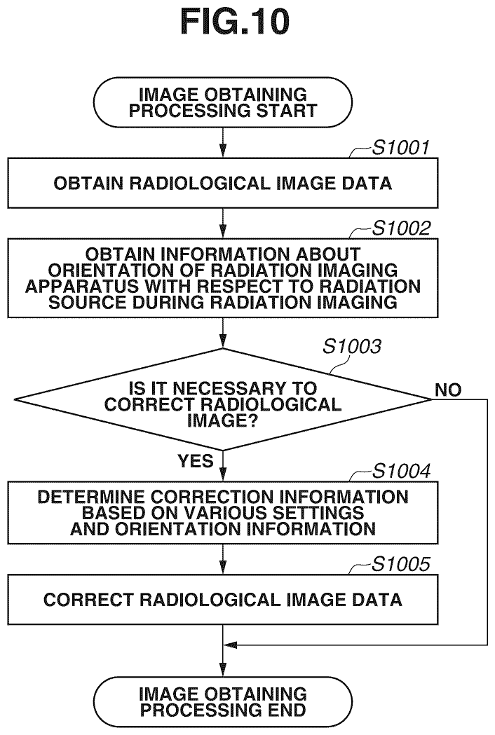

The processing of step S413 will be described in detail with reference to FIG. 10. FIG. 10 is a flowchart illustrating image obtaining processing. Each processing illustrated in this flowchart is implemented by the control unit 250 (in particular, the CPU 251) functioning as the corresponding functional unit in FIG. 3.

In step S1001, the radiological image obtaining unit 331 obtains radiological image data before correction, which is a result of integrating the doses of irradiated radiation using the image capturing pixels 221.

In step S1002, the orientation information management unit 333 reads out the orientation information held in step S902.

In step S1003, the orientation information management unit 333 determines whether it is necessary to correct the radiological image. Whether it is necessary to correct the radiological image is determined based on the presence or absence of radiological image settings by taking into account the orientation, and based on the orientation state held in step S903. For example, in a case where any one of the radiological image settings by taking into account the orientation is ON and there is an angular difference of a predetermined value or more between the orientation of the radiation source 122 and the orientation of the radiation imaging apparatus 100, it is determined that it is necessary to correct the radiological image.

In a case where the control unit 250 determines that it is necessary to correct the radiological image (YES in step S1003), the processing proceeds to step S1004. In a case where it is determined that it is not necessary to correct the radiological image (NO in step S1003), the control unit 250 terminates the processing. The radiological image obtained before correction by the radiological image obtaining unit 331 is accordingly used as it is in the subsequent-stage processing.

In step S1004, the orientation information management unit 333 transmits the orientation information to the correction unit 334. The correction unit 334 determines correction information corresponding to each pixel value in the radiological image based on the setting content in the setting information 702 and the orientation information, and transmits a notification about the correction information to the radiological image obtaining unit 331.

In step S1005, final radiological image data is generated based on the obtained correction information and the radiological image before correction.

### Advantageous Effects

As described above, the radiation imaging system 1 according to the present exemplary embodiment estimates the distance attenuation of the radiation amount depending on the direction of the radiation source 122 with respect to the radiation imaging apparatus 100, the attenuation due to a grid, and the attenuation due to the heel effect, and obtains a correction value. It is then correct an output from each image capturing pixel 221 to thereby obtain a clear radiological image. In the present exemplary embodiment, information about the difference between the orientation of the radiation source 122 and the orientation of the radiation imaging apparatus 100 is estimated by exchanging detected values from the angle sensor 106 and the angle sensor 123 via wireless communication. Thus, the difference in the orientation can be appropriately estimated even when the radiation imaging apparatus 100 is completely hidden behind a shielding object (subject) as viewed from the radiation source 122. Furthermore, a variation in the arrival amount of radiation due to the difference in the orientation and an appropriate correction method for resolving the variation can be determined.

### Second Exemplary Embodiment

To obtain a method for deriving the orientation of the radiation generation apparatus 121 and the orientation of the radiation imaging apparatus 100, each of the radiation generation apparatus 121 and the radiation imaging apparatus 100 is provided with an acceleration sensor or a gyroscope sensor to obtain an acceleration as an output value from the acceleration sensor, or an angular velocity as an output value from the gyroscope sensor, to thereby derive an orientation (e.g., position, angle).

For example, the derivation of the orientation using the gyroscope sensor is performed by integrating angular velocities at small times obtained by the gyroscope sensor. The derivation of the orientation using the acceleration sensor is performed such that accelerations obtained by the acceleration sensor are integrated once and a velocity at a certain time is derived, and then the velocity is further integrated again to thereby derive a displacement (position).

However, only the sum of variations in the orientation can be derived from the above-described method. The current orientation can be accurately derived only when an orientation to be used as a reference at a certain time (such an orientation is hereinafter referred to as a reference orientation) is known.

To avoid such a problem, in the technique discussed in PTL 2, the radiation imaging apparatus is provided with an input unit for setting a reference orientation and an alignment rule serving as a structure for positioning. The user of the radiation imaging apparatus issues an instruction to set the reference orientation (calibration processing) through the input unit when the radiation imaging apparatus is brought into contact with the alignment rule, thereby making it possible to set the reference orientation.

However, in the technique discussed in PTL 2, the user needs to issue the instruction via the input unit during setting of the reference orientation, the use procedure becomes troublesome. Further, since the integrated value is used to derive the orientation, errors will also be accumulated after a lapse of a period of time since the reference orientation is set. It is thus necessary to frequently set the reference orientation to accurately derive the orientation, which increases the time and effort for the user such as an engineer.

In the first exemplary embodiment, the angle sensor 106 included in the radiation imaging apparatus 100 and the angle sensor 123 included in the radiation source 122 are used to derive the assumed relationship between the orientation of the radiation imaging apparatus 100 and the orientation of the radiation source 122. In contrast, in a second exemplary embodiment, the assumed relationship between the orientation of the radiation imaging apparatus 100 and the orientation of the radiation source 122 is derived based on a sensor unit attached to the radiation imaging apparatus 100 and an optical image obtained by an image capturing unit. Components used in the first and second exemplary embodiments will be described below.

A radiation imaging system 10010 is a unit corresponding to the radiation imaging system 1. A radiation imaging apparatus 10100 is a unit corresponding to the radiation imaging apparatus 100. A sensor unit 10101 is a unit corresponding to the orientation detection unit 270. A relay device 10110 is a device corresponding to the communication control apparatus 110. A control apparatus 10120 is an apparatus corresponding to the information processing apparatus 150. A radiation generation apparatus 10130 is an apparatus corresponding to the radiation generation apparatus 121. A radiation source 10131 is a device corresponding to the radiation source 122. A communication device 10150 is a device corresponding to the access point 130. A control unit 10200 is a unit corresponding to the control unit 250. An orientation derivation unit 10201 is a unit corresponding to the panel orientation detection unit 332. A storage unit 10202 is a unit corresponding to the memory 252. A communication unit 10203 is a unit corresponding to the wired communication unit 280 and the wireless communication unit 290. A radiation detection unit 10205 is a unit corresponding to the radiation detector 220. A power supply generation unit 10206 is a unit corresponding to the power supply control unit 101 and the power supply control unit 260. A display unit 10223 is a unit corresponding to the display apparatus 153. An operation unit 10224 is a unit corresponding to the input apparatus 152. An orientation derivation unit 10225 is a unit corresponding to the orientation information management unit 312.

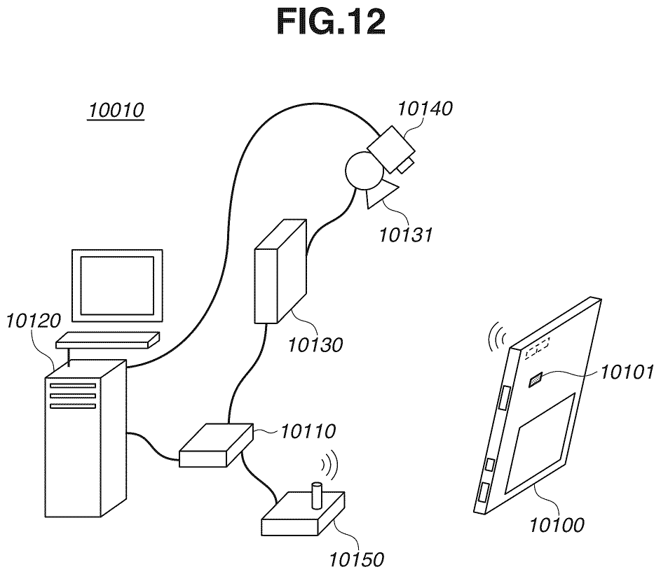

A radiation imaging system according to the second exemplary embodiment will now be described below with reference to the drawings. FIG. 12 is a schematic view illustrating the radiation imaging system according to the second exemplary embodiment.

The radiation imaging system 10010 includes the radiation imaging apparatus 10100, the relay device 10110, the radiation generation apparatus 10130, the radiation source 10131, an imaging unit 10140, and the communication device 10150 in an imaging room for capturing an image of an object (not illustrated). The control apparatus 10120 is located in an operation room for an imaging operation.

The radiation imaging apparatus 10100 can communicate with the communication device 10150 to communicate with the control apparatus 10120 via the relay device 10110. While FIG. 12 illustrates a configuration example of the radiation imaging apparatus 10100 for establishing wireless communication, wired communication can be established. In this case, the radiation imaging apparatus 10100 is connected to the relay device 10110 with a wire without involving the communication device 10150. The sensor unit 10101 is attached to the radiation imaging apparatus 10100, so that information for deriving the orientation angle and position of the radiation imaging apparatus 10100 can be output. In the following description, the orientation angle and position information is collectively referred to as “orientation information”.

The relay device 10110 has a switching hub function, and connects the radiation imaging apparatus 10100, the control apparatus 10120, and the radiation generation apparatus 10130 to a network. The relay device 10110 also has a relay function for exchanging signals for controlling a radiation exposure timing and a detection timing, for example, by transmitting operation information about the radiation generation apparatus 10130 to the radiation imaging apparatus 10100.

The control apparatus 10120 has a function for obtaining information indicating the state of the radiation imaging apparatus 10100 at a predetermined timing and displaying the information on a display or the like to transmit the information to the user. The control apparatus 10120 also includes a GUI for operating the radiation imaging apparatus 10100, thereby making it possible to control the state of the radiation imaging apparatus 10100 from the operation room. The control apparatus 10120 also captures images obtained by the imaging unit 10140. In a case where the captured images include an image of the radiation imaging apparatus 10100, the image is analyzed to derive the orientation information about the radiation imaging apparatus 10100.

The radiation generation apparatus 10130 controls irradiation of radiation from the radiation source 10131 in a preliminarily set radiation irradiation condition. To irradiate radiation, control with a GUI using pressing of a radiation irradiation switch, a display, or a touch panel is used.

An example of an object image capturing method is an image capturing method by synchronizing the radiation generation apparatus 10130 with the radiation imaging apparatus 10100. In this image capturing method, switch input information is transmitted to the radiation imaging apparatus 10100 via the relay device 10110, irradiation permission information is received from the radiation imaging apparatus 10100, and then radiation is irradiated.

The radiation generation apparatus 10130 can also receive orientation information about the radiation imaging apparatus 10100 and position and angle information relative to the radiation source 10131 from the control apparatus 10120 or the radiation imaging apparatus 10100, and can also display the information on a display device such as a display or a touch panel.

The imaging unit 10140 is an image capturing unit, such as a camera, which is attached to a portion in the vicinity of the radiation source 10131. The imaging unit 10140 is attached such that the direction in which an image is captured by the imaging unit 10140 matches the direction in which radiation is irradiated from the radiation source 10131.

Examples of the communication between the above-described units can include communication that is compliant with communication standards such as Recommended Standard (RS) 232C, a universal serial bus (USB), and Ethernet®, and communication using a dedicated signal line. This communication can be wired communication or wireless communication.

Next, an operation of each unit for image capturing using the radiation imaging system 10010 will be described.

The user turns on the radiation imaging apparatus 10100 to thereby bring the radiation imaging apparatus 10100 into an image capturing enabled state. The user adjusts the position of the object and the position of the irradiation region for radiation irradiated from the radiation source 10131. To adjust the positions, information about the orientation angle of the radiation imaging apparatus 10100 and information about the position and angle relative to the radiation source 10131 are used as auxiliary information. These pieces of information are displayed on the display of the control apparatus 10120.

The radiation generation apparatus 10130 controls the radiation source 10131 to irradiate radiation to the radiation imaging apparatus 10100 when the radiation irradiation switch is turned on. After the radiation irradiated from the radiation source 10131 is transmitted through the object, the radiation is incident on the radiation imaging apparatus 10100.

The radiation imaging apparatus 10100 generates image data corresponding to the incident radiation, and transmits the image data to the control apparatus 10120 in the operation room. The control apparatus 10120 displays the received image data. The operator of the radiation imaging system 10010 can check the image displayed on the control apparatus 10120, and determine whether it is necessary to perform image capturing again. In a case where the user determines that the displayed image is normal, the user prepares for image capturing of another object in a similar procedure.

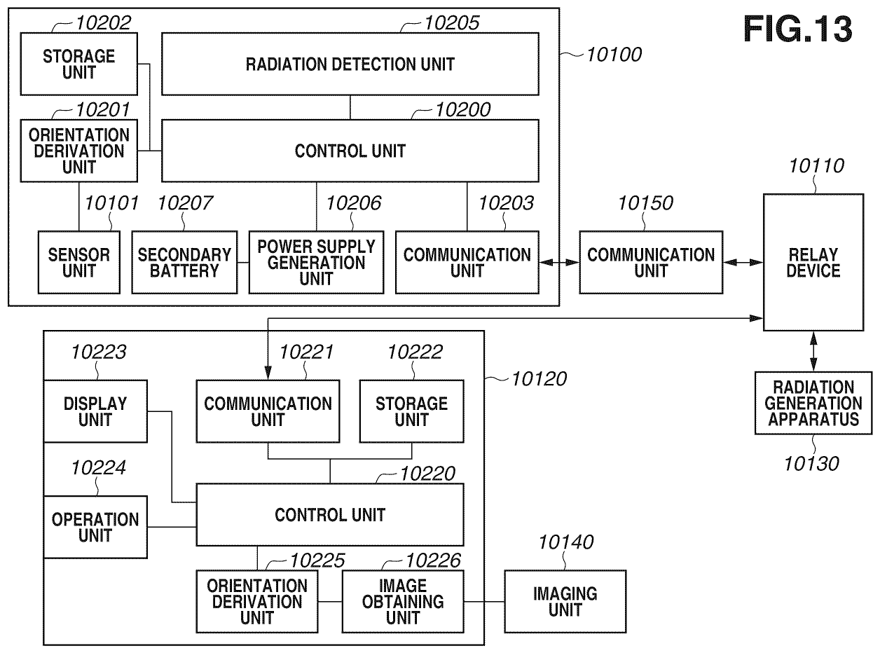

FIG. 13 is a functional block diagram of the radiation imaging system according to the present exemplary embodiment.

The radiation imaging apparatus 10100 includes the sensor unit 10101, the orientation derivation unit 10201, the control unit 10200, the storage unit 10202, the communication unit 10203, the radiation detection unit 10205, the power supply generation unit 10206, and a secondary battery 10207.

The sensor unit 10101 obtains an acceleration and an angular velocity that are serving as data for deriving the orientation angle of the radiation imaging apparatus 10100. In the present exemplary embodiment, the sensor unit 10101 is composed of a six-axis inertial measurement unit (IMU) including an acceleration sensor and a gyroscope sensor. The sensor unit 10101 can be a nine-axis IMU including a geomagnetic sensor, or a six-axis IMU including a geomagnetic sensor and a gyroscope sensor.

The orientation derivation unit 10201 derives orientation information about the radiation imaging apparatus 10100 using the acceleration and angular velocity obtained from the sensor unit 10101. The orientation information is derived from the orientation angle and position (hereinafter referred to as a reference orientation) of the radiation imaging apparatus 10100 at a certain time and information obtained from the sensor unit 10101.

The control unit 10200 controls the overall operation of the system for the radiation imaging apparatus 10100, such as driving control of the radiation detection unit 10205, digital data correction processing, and control of the communication unit 10203. The control unit 10200 is composed of, for example, a circuit board including a CPU, a graphics processing unit (GPU), a field programmable gate array (FPGA), or the like. The control unit 10200 can have the function of the orientation derivation unit 10201, or implement the functions of the control unit 10200 and the orientation derivation unit 10201 in different functional regions within one unit.

The storage unit 10202 is configured to store control programs, image data, control parameters, and operation logs for the radiation imaging apparatus 10100, and includes a non-volatile memory. While the present exemplary embodiment illustrates an example where a non-volatile memory is used, a volatile memory can also be used.

The communication unit 10203 has a function for establishing communication between the radiation imaging apparatus 10100 and another apparatus. The communication unit 10203 exchanges various information with another apparatus via wired or wireless communication.

The radiation detection unit 10205 has a function for detecting radiation irradiated from the radiation source 10131 and generating digital data (image data) corresponding to the detected radiation.

The power supply generation unit 10206 generates various power supply voltages and currents for operation of the radiation imaging apparatus 10100 from power supplied from the secondary battery 10207, and supplies power to each unit.

The secondary battery 10207 functions as a power supply for operating the above-described units. The secondary battery 10207 can be detachable, or incorporated in a casing of the radiation imaging apparatus 10100. The secondary battery 10207 can be, for example, a lithium ion battery or an electric double layer capacitor.

The control apparatus 10120 includes a control unit 10220, a communication unit 10221, a storage unit 10222, the display unit 10223, the operation unit 10224, the orientation derivation unit 10225, and an image obtaining unit 10226.

The control unit 10220 has a display control function for controlling the display of the display unit 10223. The control unit 10220 also receives operation information from the operation unit 10224, and controls the communication unit 10221 to transmit and receive signals for controlling the functions of display on the display unit 10223 and controlling the radiation imaging apparatus 10100.

The communication unit 10221 has a function for communicating with another apparatus such as the radiation imaging apparatus 10100. The communication unit 10221 exchanges various information such as operation information and captured images with another apparatus via wired communication or wireless communication.

The storage unit 10222 stores control programs for the control apparatus 10120, captured image data, control parameters, and operation logs, and includes a non-volatile memory. While the present exemplary embodiment illustrates an example where a non-volatile memory is used, a volatile memory can also be used.

The display unit 10223 includes a GUI for operating the radiation imaging apparatus 10100, and the GUI can be operated by the operation unit 10224.

The orientation derivation unit 10225 derives orientation information about the radiation imaging apparatus 10100 and the radiation source 10131 from the image obtained by the image obtaining unit 10226 from the imaging unit 10140. When it is determined that the image includes an image of the radiation imaging apparatus 10100 and orientation information can be derived, the relative positions and angles of the radiation source 10131 and the radiation imaging apparatus 10100 can be derived. The imaging unit 10140 is composed of, for example, an optical camera, and is configured to obtain moving images, still images, and the like. While the present exemplary embodiment illustrates an example where an optical camera is used, a stereo camera or the like can also be used to derive orientation information, in particular, positional information.

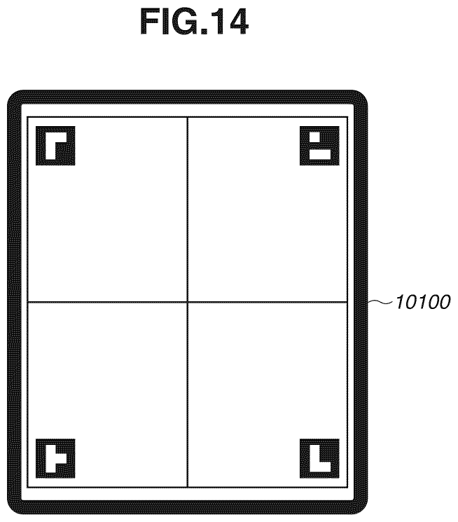

Next, a method will be described in which the orientation derivation unit 10225 derives orientation information about the radiation imaging apparatus 10100 relative to the radiation source 10131. FIG. 14 illustrates a radiation detection plane serving as a radiation incidence plane on a surface of the casing of the radiation imaging apparatus 10100. At four corners of the radiation detection plane, two-dimensional codes are printed as markers to be used for deriving the orientation information.

The image obtaining unit 10226 obtains a camera image from the imaging unit 10140, and the orientation derivation unit 10225 analyzes the size and inclination of the two-dimensional codes, thereby deriving the relative position and angle information about the radiation imaging apparatus 10100 and the radiation source 10131. In the orientation derivation unit 10225, a reference for the size and inclination of the two-dimensional codes and a reference for the position and angle relative to the radiation imaging apparatus 10100 are preliminarily set. A mounting offset for the image obtaining unit 10226 and the radiation source 10131 is preliminarily obtained. It is thus possible to derive the relative position and angle information about the radiation imaging apparatus 10100 and the radiation source 10131 from the camera image by taking into account the offset.

While FIG. 14 illustrates an example where two-dimensional codes are printed at four corners of the radiation imaging apparatus 10100, the number of two-dimensional codes is not limited to four. In the present exemplary embodiment, the two-dimensional codes are printed. Instead of using two-dimensional codes, one-dimensional barcodes, symbols, or characters can also be used.

The orientation derivation unit 10225 analyzes at least one two-dimensional code, thereby making it possible to derive orientation information. Compared to analyzing one two-dimensional code, analyzing two or more two-dimensional codes makes it likely to derive the position and angle more accurately. Thus, for example, a condition can be added in which orientation information is derived when at least two two-dimensional codes are included in the camera image.

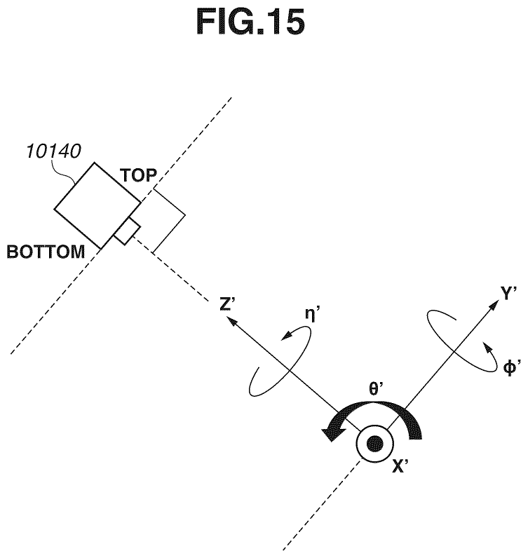

Next, orientation information to be obtained will be described with reference to FIG. 15. As illustrated in FIG. 15, coordinates of orientation information to be obtained are set assuming that directions perpendicular to the direction of the imaging unit 10140 are defined as X′ and Y′ and a direction parallel to the direction of the imaging unit 10140 is defined as Z′. This coordinate system is referred to as a camera coordinate system. Assume that a rotation about an X′ axis is referred to as roll (θ′), a rotation about a Y′ axis is referred to as pitch (φ′), and a rotation about a Z′ axis is referred to as yaw (η′).

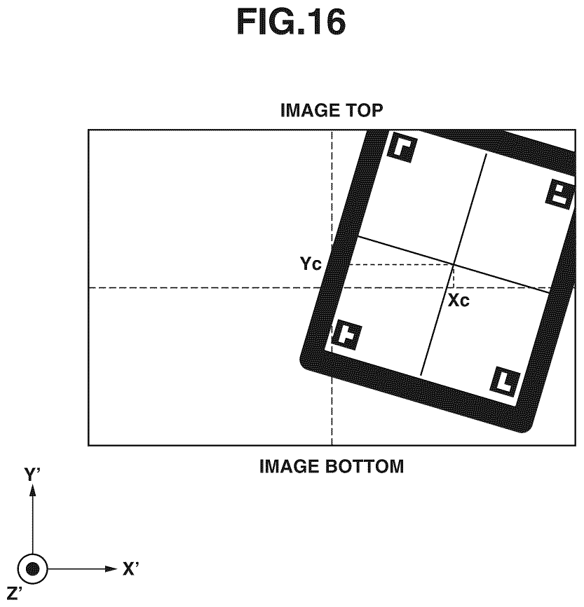

FIG. 16 illustrates an example of an image that is obtained from the imaging unit 10140 and includes an image of the radiation imaging apparatus 10100. The orientation derivation unit 10225 derives a position (X′, Y′, Z′) and rotation (θ′, φ′, η′) corresponding to a difference between a central portion of the image and the center of the radiation detection plane.

Next, a method will be described in which the orientation derivation unit 10201 derives orientation information about the radiation imaging apparatus 10100 using information obtained from the sensor unit 10101 attached to the radiation imaging apparatus 10100. The derivation of orientation information performed by the orientation derivation unit 10201 is more advantageous than the derivation of orientation information performed by the orientation derivation unit 10225. The advantage is that there is no limitation, such as the need for using an image including an image of a two-dimensional code, during the derivation, and orientation information can always be derived if the reference orientation, which is the reference for deriving the orientation information, is given.

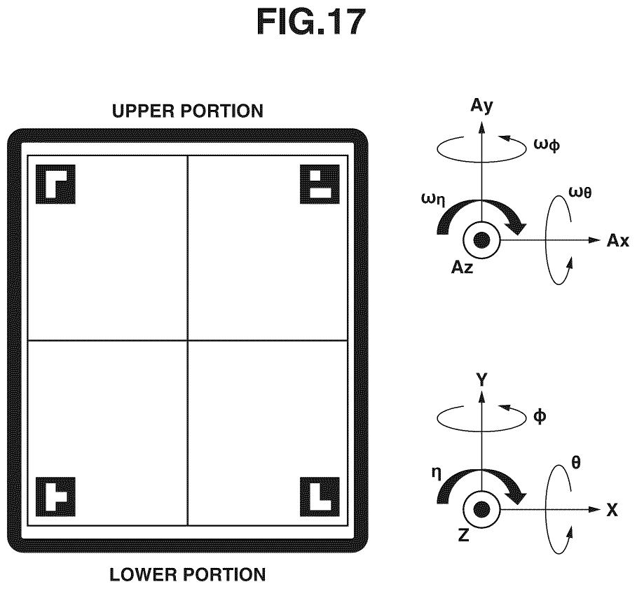

FIG. 17 illustrates a sensor coordinate system determined by the direction of the sensor. The acceleration and angular velocity obtained from the sensor unit 10101 are derived in the sensor coordinate system. In the sensor coordinate system, an upward acceleration with respect to the radiation detection plane is defined as Ay, a rightward acceleration is defined as Ax, and a vertical acceleration is defined as Az.

An angular velocity about an Ax-direction axis is represented by ωθ, an angular velocity about an Ay-direction axis is represented by ωφ, and an angular velocity about an Az-direction axis is represented by ωη. In the sensor coordinate system, a reference position is first determined and roll (θ) of rotation about the X-axis, pitch (φ) of rotation about the Y-axis, and yaw (η) of rotation about the Z-axis in the directions X, Y, and Z are determined, respectively. At the reference position, (X, Y, Z, θ, φ, η)=(0, 0, 0, 0, 0, 0) holds. This is referred to as a reference coordinate system.

In a case where the orientation derivation unit 10201 derives orientation information about the radiation imaging apparatus 10100, the orientation information to be derived is obtained by adding the amount of movement corresponding to the integrated value of the acceleration and the angular velocity, to the reference orientation. The reference orientation is represented by {x(0), y(0), z(0), θ(0), φ(0), η(0)}, and the angle at time t after setting the reference orientation is obtained assuming that an angular velocity measurement time interval is represented by Δt and a measurement count is represented by n(t=nΔt). In this case, the roll, the pitch, and the yaw at time t can be obtained by the following formulas.

$$θ ( t ) = θ ( 0 ) + {\sum }_{k = 1}^{n} {ω}_{θ} ( k Δ t ) Δ t \qquad ( 1 )$$

$$φ ( t ) = φ ( 0 ) + {\sum }_{k = 1}^{n} {ω}_{φ} ( k Δ t ) Δ t \qquad ( 2 )$$

$$η ( t ) = η ( 0 ) + {\sum }_{k = 1}^{n} {ω}_{η} ( k Δ t ) Δ t \qquad ( 3 )$$

Similarly, the roll, the pitch, and the yaw at the positions X, Y, and Z can also be derived. Assuming that x-axis components of the position, velocity, and acceleration at time t are represented by x(t), vₓ(t), and aₓ(t), respectively, y-axis components thereof are represented by y(t), v_{y}(t), and a_{y}(t), respectively, and z-axis components thereof are represented by z(t), v_{z}(t), and a_{z}(t), respectively, the velocity at time t can be derived from the following formulas.

$${v}_{x} ( t ) = {v}_{x} ( 0 ) + {\sum }_{k = 1}^{n} {a}_{x} ( k Δ t ) Δ t \qquad ( 4 )$$

$${ν}_{y} ( t ) = {v}_{y} ( 0 ) + {\sum }_{k = 1}^{n} {a}_{y} ( k Δ t ) Δ t \qquad ( 5 )$$

$${v}_{z} ( t ) = {v}_{z} ( 0 ) + {\sum }_{k = 1}^{n} {a}_{z} ( k Δ t ) Δ t \qquad ( 6 )$$

Thus, the positions at time t can be derived from the following formulas.

$$x ( t ) = x ( 0 ) + {\sum }_{k = 1}^{n} {v}_{x} ( k Δ t ) Δ t \qquad ( 7 )$$

$$y ( t ) = y ( 0 ) + {\sum }_{k = 1}^{n} {v}_{y} ( k Δ t ) Δ t \qquad ( 8 )$$

$$z ( t ) = z ( 0 ) + {\sum }_{k = 1}^{n} {v}_{z} ( k Δ t ) Δ t \qquad ( 9 )$$

Thus, the positions can be obtained by the formulas described above.

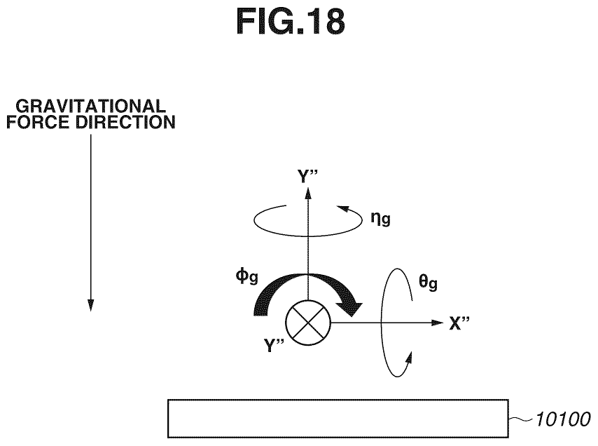

To derive the velocity and position, it may be desirable to subtract the effect of a gravitational force on the acceleration. As illustrated in FIG. 18, when the angle in the direction of gravitational force set as an opposite direction of the Z-axis (hereinafter referred to as a gravitational force coordinate system) is set as a reference angle “0”, the rotational angles about the respective axes are represented by roll (θg), pitch (φg), and yaw (ηg), respectively. In this case, an acceleration (a_{gx}, a_{gy}, a_{gz}) due to the gravitational force can be given by the following formula.

$$[ \begin{matrix}{a}_{gx} \\ {a}_{gy} \\ {a}_{gz}\end{matrix} ] = [ \begin{matrix}g sin {θ}_{g} \\ - g sin {φ}_{g} cos {θ}_{g} \\ - g cos {φ}_{g} cos {θ}_{g}\end{matrix} ] \qquad ( 10 )$$

θ_{g} and φ_{g} can be obtained assuming that t=0 and θ(0) and φ(0) are set to “0” in the state illustrated in FIG. 18 by formulas (1) and (2).

θ_{g} and φ_{g} can be obtained by the following formulas (11) and (12) when the radiation imaging apparatus 10100 is in a stationary state.

$${θ}_{g} = {tan}^{- 1} \frac{Ax}{\sqrt{{A}_{y}^{2} + {A}_{z}^{2}}} \qquad ( 11 )$$

$${φ}_{g} = {tan}^{- 1} \frac{{A}_{y}}{\sqrt{{A}_{x}^{2} + {A}_{z}^{2}}} \qquad ( 12 )$$

Since the rotation amounts at time t are integrated in formulas (1) and (2), the error is also accumulated when an error occurs, which leads to a reduction in accuracy. In formulas (11) and (12), the direction of gravitational force on the acceleration sensor in the stationary state is used for derivation, so that time t has little influence.

θ(0) and φ(0) when reference value t=0 in the stationary state can also be derived from formulas (11) and (12), and the calculation results can be applied to formulas (1) and (2). In this case, the angles derived from formulas (11) and (12) are updated as θ(0) and φ(0) when the reference value t=0 every time the stationary state is determined. The use of this method makes it possible to reset the accumulated errors that may cause deterioration in the accuracy in formulas (1) and (2), thereby improving the accuracy.

In each of formulas (4) to (6), aₓ, a_{y}, and a_{z} represent a reference coordinate system, and the acceleration in the sensor coordinate system obtained from the sensor unit 10101 cannot be used as it is. It may thus be desirable to convert the acceleration (aₒᵤₜₓ, a_{outy}, a_{outz}) in the sensor coordinate system obtained by the sensor unit 10101 at time t into the reference coordinate system.

Assuming that a conversion matrix for each axis is represented by Rₓ, R_{y}, and R_{z}, the following formulas can be obtained.

$$Rx ( θ ) = [ \begin{matrix}1 & 0 & 0 \\ 0 & cos θ & sin θ \\ 0 & - sin θ & cos θ\end{matrix} ] \qquad ( 13 )$$

$$Ry ( φ ) = [ \begin{matrix}cos φ & 0 & - sin φ \\ 0 & 1 & 0 \\ sin φ & 0 & cos φ\end{matrix} ] \qquad ( 14 )$$

$$Rz ( η ) = [ \begin{matrix}cos η & sin η & 0 \\ - sin η & cos η & 0 \\ 0 & 0 & 1\end{matrix} ] \qquad ( 15 )$$

The acceleration (aₒᵤₜₓ, a_{outy}, a_{outz}) output from the sensor coordinate system is obtained by rotating the reference coordinate system by θ(t), φ(t), and η(t) with respect to the reference coordinate system. To restore the reference coordinate system, it may thus be desirable to rotate the coordinate system by −θ(t), −φ(t), and −η(t). Aₓ, a_{y}, a_{z} can thus be obtained by the following formula.

$$[ \begin{matrix}{a}_{x} \\ {a}_{y} \\ {a}_{z}\end{matrix} ] = Rz ( - η ( t ) ) R y ( - φ ( t ) ) R x ( - θ ( t ) ) [ \begin{matrix}{a}_{outx} - {a}_{gx} \\ {a}_{outy} - {a}_{gy} \\ {a}_{outz} - {a}_{gz}\end{matrix} ] \qquad ( 16 )$$

The position in the reference coordinate system can be derived using this acceleration.

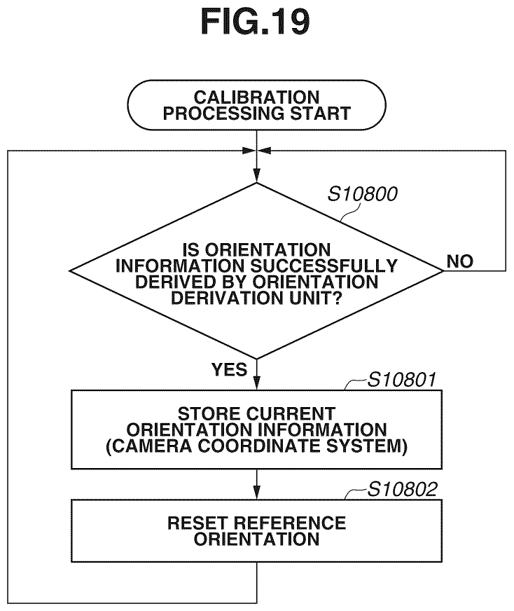

Next, a procedure for calibration processing for the reference orientation will be described with reference to FIG. 19.

In step S10800, it is determined whether the orientation derivation unit 10225 has successfully derived the orientation information. For example, the orientation information can be derived when the camera image obtained by the imaging unit 10140 includes an image of a two-dimensional code printed on the radiation imaging apparatus 10100. In a case where the orientation information has been successfully derived (YES in step S10800), the processing proceeds to step S10801. In a case where the orientation information cannot be derived because, for example, the image of the two-dimensional code had not been captured, the processing of step S10800 is performed again. The cycle of performing the processing of step S10800 again can be arbitrarily set.

In step S10801, the orientation derivation unit 10225 stores relative orientation information about the radiation source 10131 and the radiation imaging apparatus 10100 derived from the camera image in the storage unit 10202. The camera coordinate system is used for the coordinates in this case.

In step S10802, the reference orientation to be used for the orientation derivation unit 10201 to derive the orientation of the radiation imaging apparatus 10100 is set using the orientation information derived in step S10801. In this case, the position of the reference orientation in the sensor coordinate system is matched with that in the camera coordinate system. In other words, the position where the camera and the sensor are located at the same position is set as the position of the reference orientation.

The processing of step S10800 to be performed again after setting the reference orientation is performed, for example, every 100 ms (milliseconds). This period can be arbitrarily set. A short cycle is desirably set because the amount of errors to be accumulated is small, so that the accuracy of orientation information can be improved.

The above-described procedure makes it possible to perform calibration processing on the reference orientation of the radiation imaging apparatus 10100 using the information derived by the orientation derivation unit 10225.

In this case, the displacement amount of the angle during a period from time 0 to time t can be derived from formulas (1), (2), and (3). A relative angle (θ′, φ′, η′) between the radiation source 10131 and the radiation imaging apparatus 10100 can be derived from the following formulas using formulas (1) to (3) and the relative angle information set in step S10802.
θ′=θ(t)
φ′=φ(t)
η′=η(t)

Similarly, a relative position (X′, Y′, Z′) is derived from the following formulas using formulas (7), (8), and (9) and the relative position information set in step S10802.
X′=x(t)
Y′=y(t)
Z′=z(t)

The above-described procedure enables the radiation imaging apparatus 10100 to derive the relative orientation information about the radiation source 10131. The orientation information is transmitted to the control apparatus 10120 or the radiation generation apparatus 10130, thereby transmitting the orientation information to the user via a display device or the like, so that the user can use the orientation information during alignment between the radiation imaging apparatus 10100 and the radiation source 10131.

While the present exemplary embodiment described above illustrates an example where the orientation information about the radiation imaging apparatus 10100 is derived by the orientation derivation unit 10201, the present exemplary embodiment is not limited to this example. For example, the orientation information can be derived by the orientation derivation unit 10225 while an image of a two-dimensional code is being captured by the imaging unit 10140. However, in the case of performing radiation imaging, two-dimensional codes can be hidden behind an object in many cases. Accordingly, the derivation of the orientation performed by the orientation derivation unit 10201 in the procedure according to the present exemplary embodiment is advantageous in that the orientation can be derived with high accuracy without paying any attention to the fact that two-dimensional codes can be hidden behind an object.

According to the present exemplary embodiment described above, the imaging unit 10140 captures an image of the radiation imaging apparatus 10100, thereby making it possible to easily perform calibration processing on the reference orientation of the sensor unit 10101. Further, calibration processing using the captured image makes it possible to easily increase the frequency of calibration processing, thereby improving the accuracy of orientation information derived in the radiation imaging apparatus 10100. Furthermore, since calibration processing can be easily achieved, the time and effort for the user can be reduced.

While the present exemplary embodiment described above illustrates an example where camera images are periodically obtained in calibration processing and orientation information can be derived, the present exemplary embodiment is not limited to this example. For example, an engineer can perform calibration processing by moving a camera image in advance such that an image of a two-dimensional barcode can be captured every time radiation imaging is performed.

Further, the accuracy of calibration processing can deteriorate in a case where the radiation imaging apparatus 10100 and the radiation source 10131 are not in a stationary state. Thus, it can be determined whether the radiation imaging apparatus 10100 and the radiation source 10131 are in a stationary state, and the calibration processing can be performed only when the radiation imaging apparatus 10100 and the radiation source 10131 are in a stationary state. In this case, the determination as to whether the radiation imaging apparatus 10100 and the radiation source 10131 are in a stationary state can be made by comparing camera images captured at successive times, by using values from the sensor unit 10101, or by using any other known sensors or the like.

### Third Exemplary Embodiment

An orientation information coordinate system to be handled in calibration processing according to a third exemplary embodiment is different from that according to the second exemplary embodiment. The third exemplary embodiment will be described below with reference to the drawings.

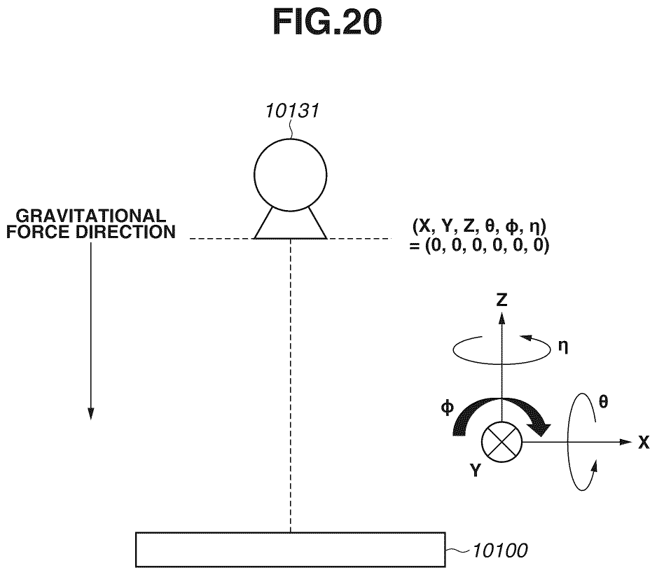

In a coordinate system according to the third exemplary embodiment, a home position of the radiation source 10131 is set as an origin. FIG. 20 illustrates an example of how to take the home position of the radiation source 10131. As illustrated in FIG. 20, the radiation irradiation direction and the direction of gravitational force are set to be coincident with each other, and a gravitational force coordinate system is used in which a direction parallel to the direction of gravitational force is defined as a Z-axis and directions parallel to the direction of gravitational force are defined as an X-axis and a Y-axis. Assuming that θ, φ, and η are defined as rotational directions about the respective axes, (X, Y, Z, θ, φ, η)=(0, 0, 0, 0, 0, 0) holds at the home position.

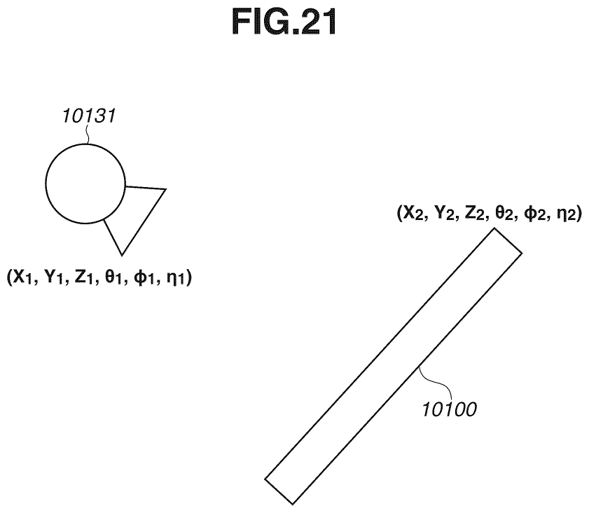

Next, when the radiation source 10131 and the radiation imaging apparatus 10100 are moved, orientation information about the radiation source 10131 and orientation information about the radiation imaging apparatus 10100 are represented by (X₁, Y₁, Z₁, θ₁, φ₁, η₁) and (X₂, Y₂, Z₂, θ₂, φ₂, η₂), respectively, as illustrated in FIG. 21.

In this case, it may be preferable to mount a component from which a position and an angle can be obtained, such as an encoder, in the radiation generation apparatus 10130 so that orientation information (X₁, Y₁, Z₁, θ₁, φ₁, η₁) about the radiation source 10131 can be obtained. The present exemplary embodiment is not limited to this example. Like in the radiation imaging apparatus 10100, orientation information can be derived using values measured by an attached six-axis IMU sensor, or can be derived by using any other known method.

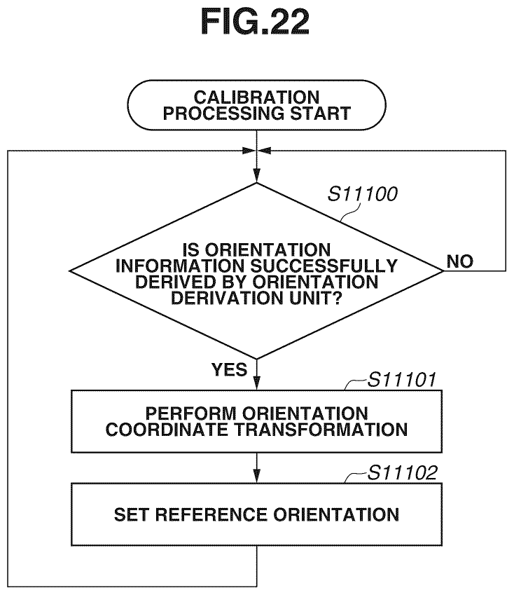

The orientation information (X₂, Y₂, Z₂, θ₂, φ₂, η₂) about the radiation imaging apparatus 10100 is derived by the orientation derivation unit 10201. A procedure for calibration processing on the reference orientation for the orientation derivation unit 10201 to derive the orientation information will be described below with reference to FIG. 22.

The calibration processing is performed in the procedure illustrated in FIG. 22. In step S11100, the orientation derivation unit 10225 determines whether the orientation information has been derived. In a case where the orientation information has been derived (YES in step S11100), the processing proceeds to step S11101. In a case where the orientation information has not been derived (NO in step S11100), the processing of step S11100 is performed again. Any cycle period for performing the processing of step S11100 again can be set.

In step S11101, coordinate conversion of the orientation information derived by the orientation derivation unit 10225 is performed. As described above in the second exemplary embodiment, the orientation derivation unit 10225 can derive relative orientation information about the radiation source 10131 and the radiation imaging apparatus 10100. In the present exemplary embodiment, the relative orientation information about the radiation source 10131 and the radiation imaging apparatus 10100 is converted into the gravitational force coordinate system illustrated in FIG. 20. When the relative orientation information derived by the orientation derivation unit 10225 is represented by (X′, Y′, Z′, θ′, φ′, η′), the difference (X, Y, Z) in the orientation information converted into the gravitational force coordinate system can be given by the following formula using the rotation matrices in formulas (13) to (15).

$$[ \begin{matrix}X \\ Y \\ Z\end{matrix} ] = Rz ( - {η}_{1} ) R y ( - {φ}_{1} ) Rx ( - {θ}_{1} ) [ \begin{matrix}{X}^{′} \\ {Y}^{′} \\ {Z}^{′}\end{matrix} ] \qquad ( 17 )$$

In step S11102, the orientation information about the radiation imaging apparatus 10100 derived from the orientation derivation unit 10225 is set as a reference orientation for the orientation derivation unit 10201. Orientation information (X₂, Y₂, Z₂, θ₂, φ₂, η₂) about the radiation imaging apparatus 10100 is given by the following formulas.

$${X}_{2} = {X}_{1} + X$$

$${Y}_{2} = {Y}_{1} + Y$$

$${Z}_{2} = {Z}_{1} + Z$$

$${θ}_{2} = {θ}_{1} + {θ}^{′}$$

$${φ}_{2} = {φ}_{1} + {φ}^{′}$$

$${η}_{2} = {η}_{1} + {η}^{′}$$

The derived orientation information (X₂, Y₂, Z₂, θ₂, φ₂, η₂) about the radiation imaging apparatus 10100 is set as the reference orientation when t=0 in the gravitational force coordinate system.

After the reference orientation is set, the orientation derivation unit 10201 can derive orientation information (X2t, Y2t, Z2t, θ2t, φ2t, η2t) about the radiation imaging apparatus 10100 at time t from formulas (1) to (16) described in the second exemplary embodiment.

The above-described procedure is performed and the radiation imaging apparatus 10100 transmits the current orientation information to the control apparatus 10120, thereby enabling the control apparatus 10120 to derive the relative orientation information about the radiation imaging apparatus 10100 and the radiation source 10131. Orientation information (X1t, Y1t, Z1t, θ1t, φ1t, η1t) about the radiation source 10131 at time t can be recognized by the control apparatus 10120 based on information from encoder components or the like. Thus, relative orientation (Xt′, Yt′, Zt′, θt′, φt′, ηt′) can be derived from the following formulas by taking the difference between orientation information about the radiation imaging apparatus 10100 and orientation information about the radiation source 10131.

$$X {t}^{′} = X 2 t - X 1 t$$

$$Y {t}^{′} = Y 2 t - Y 1 t$$

$$Z {t}^{′} = Z 2 t - Z 1 t$$

$$θ {t}^{′} = θ 2 t - θ 1 t$$

$$φ {t}^{′} = φ 2 t - φ 1 t$$

$$η {t}^{′} = η 2 t - η 1 t$$

The configuration of the orientation information coordinate system to be handled in the calibration processing according to the present exemplary embodiment that is different from the first exemplary embodiment has been described above. Further, the values output from the orientation derivation unit 10201 of the radiation imaging apparatus 10100 are also included in orientation information from the home position. The imaging unit 10140 captures an image of the radiation imaging apparatus 10100, thereby making it possible to easily perform calibration processing on the sensor unit 10101.

The calibration processing using the captured image makes it possible to easily increase the frequency of calibration processing, thereby improving the accuracy of orientation information derived in the radiation imaging apparatus 10100. Furthermore, since the calibration processing can easily be achieved, the time and effort for the user can be reduced.

### Other Exemplary Embodiment

The present invention is not limited to the above-described exemplary embodiments and can be modified in various ways based on the gist of the present invention. Such modifications cannot be excluded from the scope of the present invention. That is, all combinations of configurations according to the above-described exemplary embodiments and modified examples thereof are also included in the present invention.

The first exemplary embodiment described above illustrates a configuration example where a radiation imaging apparatus that obtains a radiological image is integrated with a radiation detection apparatus that detects a radiation dose. However, the radiation imaging apparatus and the radiation detection apparatus can be separate from each other as long as radiation can be detected and auto exposure control can be performed.

The first exemplary embodiment described above illustrates a configuration example where the radiation imaging apparatus 100 includes the correction unit 334. However, image correction can be performed at any other location. For example, image correction can be performed in the information processing apparatus 150.

The first exemplary embodiment described above illustrates processing for correcting pixel values for dose detection pixels to perform auto exposure control. However, any other method can be used as long as the auto exposure control timing can be appropriately corrected. For example, the threshold for integration of doses to be used for auto exposure control can be controlled based on the orientation information about the radiation source and the orientation information about the radiation imaging apparatus to thereby correct the auto exposure control timing.

Further, the dose detection region to be used for auto exposure control in the plurality of dose detection regions can be changed based on the orientation information about the radiation source and the orientation information about the radiation imaging apparatus to thereby correct the auto exposure control timing. A logic obtained by combining a plurality of dose detection regions can also be changed based on the orientation information about the radiation source and the orientation information about the radiation imaging apparatus. Specifically, a logic that irradiation of radiation is stopped when one of three dose detection regions reaches a threshold can be changed to a logic that the irradiation is stopped when all the three regions reach the threshold.

In the first exemplary embodiment, correction processing to resolve the variation in the arrival amount of radiation due to the orientation (angle) of the radiation imaging apparatus with respect to the radiation source has been described above. However, the variation in the arrival amount of radiation to be resolved can be caused due to a positional relationship of the radiation imaging apparatus relative to the radiation source. FIG. 11E illustrates a distribution of the arrival amount of radiation in a case where the radiation source faces the center of the radiation imaging apparatus. FIG. 11F illustrates a distribution of the arrival amount of radiation in a case where the radiation source does not face the center of the radiation imaging apparatus. In a case where the radiation source faces the center of the radiation imaging apparatus as illustrated in FIG. 11E, the arrival amount of radiation at the center of the radiation imaging apparatus at a short distance tends to increase, and the arrival amount of radiation at an end of the radiation imaging apparatus at a long distance tends to decrease. In contrast, in a case where the radiation source does not face the center of the radiation imaging apparatus as illustrated in FIG. 11E, the arrival amount of radiation at one end of the radiation imaging apparatus at a short distance tends to increase. In contrast, the arrival amount of radiation at the center of the radiation imaging apparatus at a long distance tends to decrease, and the arrival amount of radiation at another end of the radiation imaging apparatus at a long distance tends to decrease. This tendency can be taken into account in various correction processing described above.

In this case, a technique such as ultra wideband (UWB) or Bluetooth® Low Energy with which the distance and angle can be detected can be used to obtain the positional relationship (positional relationship information) between the radiation source and the radiation imaging apparatus. The radiation source and the radiation imaging apparatus can be provided with a UWB module or a Bluetooth® Low Energy module and can communicate with each other via wireless communication, thereby obtaining information about the relative positional relationship.

The present invention can also be implemented by processing in which a program for implementing one or more functions according to the exemplary embodiments described above is supplied to a system or an apparatus via a network or a storage medium, and one or more processors in a computer of the system or the apparatus read out and execute the program. The present invention can also be implemented by a circuit for implementing one or more functions according to the exemplary embodiments described above.

Examples of the processor or the circuit can include a CPU, a micro processing unit (MPU), a GPU, an application-specific integrated circuit circuit (ASIC), and an FPGA. Examples of the processor or the circuit may also include a digital signal processor (DSP), a data flow processor (DFP), and a neural processing unit (NPU).

The radiation imaging system according to the above-described exemplary embodiments can be implemented as a single apparatus, or can be implemented as a configuration for executing the above-described processing using a combination of a plurality of apparatuses so that the apparatuses can communicate with each other. Such modifications are also included in the exemplary embodiments of the present invention. The above-described processing can be executed by a common server apparatus or a server group. The plurality of apparatuses constituting the radiation imaging system can be configured to establish communication at a predetermined communication rate, and need not necessarily be located in the same facility or in the same country.

The exemplary embodiments of the present invention may include a configuration for performing processing in which a software program for implementing the functions according to the above-described exemplary embodiments is supplied to a system or an apparatus and a computer in the system or the apparatus reads out and executes a program code.

Thus, the program code to be installed on a computer for the computer to implement the processing according to the exemplary embodiments is one of the exemplary embodiments of the present invention. The present invention can also be implemented by processing in which an operating system (OS) or the like that runs on the computer performs a part or all of the actual processing based on an instruction included in the program read out by the computer, thereby making it possible to implement the functions according to the exemplary embodiments described above. Furthermore, the program code read out from a storage medium can be written into a memory that is included in a function extension board incorporated in the computer or is included in a function extension unit connected to the computer. The present invention can also be implemented by processing in which a CPU or the like included in the function extension board or the function extension unit performs a part or all of the actual processing based on an instruction in the program code, thereby implementing the above-described functions.

As for the exemplary embodiments described above, the following supplementary notes are disclosed as one aspect and selective features of the invention.

### Supplementary Note 1

A radiation imaging apparatus for performing radiation imaging based on irradiated radiation, the radiation imaging apparatus including:
a radiation detection unit configured to detect radiation;
a sensor unit configured to output information about an orientation angle and a position change of the radiation imaging apparatus; and
an orientation derivation unit configured to derive the orientation angle and the position based on information about the position change and information based on a captured image of the radiation imaging apparatus obtained by an image capturing unit included in a radiation imaging system.

### Supplementary Note 2

The orientation derivation unit can set a reference orientation serving as a reference for derivation of the orientation angle and the position from information based on the image, and can derive the orientation angle and the position from the reference orientation and the change information.

### Supplementary Note 3

The reference orientation can be obtained based on the orientation angle and the position of a radiation source included in the radiation imaging system.

### Supplementary Note 4

The sensor unit can include at least an acceleration sensor and a gyroscope sensor.

### Supplementary Note 5

A radiation imaging system including:
a radiation imaging apparatus according to any one of Supplementary Notes 1 to 4;
a control apparatus configured to control the radiation imaging apparatus; and
the image capturing unit.

### Supplementary Note 6

The control apparatus can derive a reference orientation based on which the orientation angle and the position are derived, from information based on the image.

### Supplementary Note 7

The radiation imaging apparatus includes a plurality of markers on a surface of the radiation imaging apparatus, and in a case where two or more of the plurality of markers are included in the image, the control apparatus can derive the reference orientation based on the image during image capturing with the image capturing unit.

### Supplementary Note 8

Each of the markers can be a two-dimensional code.

### Supplementary Note 9

The control apparatus can cause the image capturing unit to perform the image capturing in a case where the radiation imaging apparatus is in a stationary state.

### Supplementary Note 10

A control apparatus for a radiation imaging system including a radiation imaging apparatus configured to perform radiation imaging based on irradiated radiation, the control apparatus being configured to set, in the radiation imaging apparatus, a reference orientation based on which an orientation angle and a position of the radiation imaging apparatus are derived, from information based on a captured image of the radiation imaging apparatus obtained by an image capturing unit included in the radiation imaging system.

### Supplementary Note 11

A control method for a radiation imaging system including a radiation imaging apparatus configured to perform radiation imaging based on irradiated radiation, the control method including:
an image capturing step of capturing an image of the radiation imaging apparatus obtained by an image capturing unit included in the radiation imaging system;
a derivation step of deriving a reference orientation serving as a reference for deriving an orientation angle and a position of the radiation imaging apparatus from information based on the image obtained by the image capturing; and
a setting step of setting the reference orientation in the radiation imaging apparatus.

### Supplementary Note 12

A radiation detection apparatus that detects radiation irradiated from a radiation source, the radiation detection apparatus including:
a detector configured to detect a radiation dose;
a unit configured to execute communication processing to stop irradiation of radiation from the radiation source in a case where a detection status of the detector satisfies a predetermined condition;
a unit configured to obtain first orientation information corresponding to an orientation of the detector and second orientation information corresponding to an orientation of the radiation source; and
a unit configured to correct the predetermined condition based on at least the first orientation information and the second orientation information.

### Supplementary Note 13

The radiation detection apparatus according to Supplementary Note 12, in which the predetermined condition is a condition that a cumulative value of values obtained by performing correction processing on a dose value obtained from the detector based on the first orientation information and the second orientation information satisfies a predetermined value.

### Supplementary Note 14

The radiation detection apparatus according to Supplementary Note 13, in which the correction processing includes processing of calibrating an attenuation of a radiation amount due to a grid.

### Supplementary Note 15

The radiation detection apparatus according to Supplementary Note 13, in which the correction processing includes processing of calibrating an attenuation of a radiation amount due to a heel effect of the radiation source.

### Supplementary Note 16

The radiation detection apparatus according to Supplementary Note 12, in which the predetermined condition is a condition that a cumulative value of values obtained by performing correction processing on a dose value obtained from the detector based on the first orientation information and the second orientation information satisfies a predetermined value.

### Supplementary Note 17

The radiation detection apparatus according to Supplementary Note 12, in which the unit configured to correct the predetermined condition is a unit configured to correct at least one parameter forming the predetermined condition, and the at least one parameter indicates any one of a cumulative dose threshold for determining whether to stop irradiation of the radiation, a cumulative dose determination method for determining whether to stop irradiation of the radiation, a radiation detection region where a radiation dose is to be monitored, a cumulative dose in the radiation detection region to be monitored, and an average value of cumulative doses in the radiation detection region to be monitored.

### Supplementary Note 18

The radiation detection apparatus according to Supplementary Note 12, in which the detector includes a plurality of dose detection pixels arrayed to form a plurality of rows and a plurality of columns.

### Supplementary Note 19

The radiation detection apparatus according to Supplementary Note 18, in which the detector further includes a plurality of image capturing pixels for outputting a radiological image corresponding to radiation, and the radiation detection apparatus further includes a unit configured to obtain a corrected radiological image based on the radiological image obtained from the plurality of image capturing pixels, the first orientation information, and the second orientation information.

### Supplementary Note 20

The radiation detection apparatus according to Supplementary Note 12, further including an angle sensor configured to obtain the first orientation information, in which the angle sensor obtains angular information using at least one of an acceleration and magnetism.

### Supplementary Note 21

The radiation detection apparatus according to Supplementary Note 12, in which the second orientation information is information obtained from another angle sensor included in the radiation source, and the other angle sensor obtains angular information using at least one of an acceleration and magnetism.

### Supplementary Note 22

The radiation detection apparatus according to Supplementary Note 12, further including a unit configured to obtain positional relationship information indicating a relative positional relationship with the radiation source, in which the unit configured to correct the predetermined condition corrects the predetermined condition based on at least the first orientation information, the second orientation information, and the positional relationship information.

### Supplementary Note 23

The radiation detection apparatus according to Supplementary Note 12, further including a unit configured to obtain positional relationship information indicating a relative positional relationship with the radiation source, in which the unit configured to correct the predetermined condition corrects the predetermined condition based on at least the first orientation information, the second orientation information, and the positional relationship information.

### Supplementary Note 24

A radiation detection system that causes a radiation detection apparatus to detect radiation irradiated from a radiation source, the radiation detection system including:
a detector configured to detect a radiation dose;
a unit configured to execute communication processing to stop irradiation of radiation from the radiation source in a case where a detection status of the detector satisfies a predetermined condition;
a unit configured to obtain first orientation information corresponding to an orientation of the detector and second orientation information corresponding to an orientation of the radiation source; and
a unit configured to correct the predetermined condition based on at least the first orientation information and the second orientation information.

### Supplementary Note 25

The radiation detection system according to Supplementary Note 24, further including a display unit configured to display a screen for designating a parameter to be corrected by the unit configured to correct the predetermined condition.

### Supplementary Note 26

The radiation detection system according to Supplementary Note 24, further including a display unit configured to display a screen based on at least the first orientation information and the second orientation information.

### Supplementary Note 27

The radiation detection system according to Supplementary Note 26, in which the screen is a screen for transmitting a notification indicating that the radiation source and the radiation detection apparatus have a predetermined orientation relationship.

### Supplementary Note 28

The radiation detection system according to Supplementary Note 24, further including a unit configured to inhibit start of irradiation of radiation from the radiation source based on at least the first orientation information and the second orientation information.

### Supplementary Note 29

A radiation imaging apparatus that performs radiation imaging based on radiation irradiated from a radiation source, the radiation imaging apparatus including:
a detector configured to obtain a radiological image based on radiation;
a unit configured to obtain first orientation information corresponding to an orientation of the detector and second orientation information corresponding to an orientation of the radiation source; and
a unit configured to correct the obtained radiological image based on at least the first orientation information and the second orientation information.

### Supplementary Note 30

A radiation detection system including a radiation source configured to irradiate radiation and a radiation detection apparatus configured to detect the radiation, the radiation detection system including:
an optical image capturing unit configured to obtain an optical image by capturing an image of the radiation detection apparatus;
a first obtaining unit configured to obtain information about an orientation angle change and information about a position change output from a sensor unit included in the radiation detection apparatus;
a second obtaining unit configured to obtain information about a reference orientation angle and information about a reference position of the radiation detection apparatus based on the optical image; and
a third obtaining unit configured to obtain information about an orientation angle and information about a position of the radiation detection apparatus at a predetermined timing based on the information about the reference orientation angle, the information about the reference position, the information about the orientation angle change, and the information about the position change.

### Supplementary Note 31

The radiation detection system according to Supplementary Note 30, in which the reference orientation angle and the reference position respectively correspond to an angle and a position relative to the radiation source.

### Supplementary Note 32

The radiation detection system according to Supplementary Note 30 or 31, in which the sensor unit includes at least an acceleration sensor and a gyroscope sensor.

### Supplementary Note 33

The radiation detection system according to any one of Supplementary Notes 30 to 32, further including a control apparatus including the first obtaining unit, the second obtaining unit, and the third obtaining unit.

### Supplementary Note 34

The radiation detection system according to Supplementary Note 33, in which the radiation imaging apparatus includes a plurality of markers on a surface of the radiation imaging apparatus, and in a case where two or more of the plurality of markers are included in the optical image, the control apparatus derives the reference orientation angle and the reference position based on the optical image.

### Supplementary Note 35

The radiation detection system according to Supplementary Note 33 or 34, in which each of the markers is a two-dimensional code.

### Supplementary Note 36

The radiation detection system according to any one of Supplementary Notes 33 to 35, in which the control apparatus includes a unit configured to detect a stationary state of the radiation detection apparatus based on an output from the sensor unit or a plurality of optical images, and information about an orientation angle and information about a position of the radiation detection apparatus at a predetermined timing are obtained based on the output from the sensor unit and the optical image in a state where the radiation detection apparatus is in the stationary state.

### Supplementary Note 37

The radiation detection system according to any one of Supplementary Notes 33 to 36, further including a unit configured to execute correction processing based on the information about the orientation angle and the information about the position at the predetermined timing.

### Supplementary Note 38

The radiation detection system according to Supplementary Note 37, in which the correction processing includes processing of calibrating an attenuation of a radiation amount due to a grid.

### Supplementary Note 39

The radiation detection system according to Supplementary Note 37 or 38, in which the correction processing includes processing of calibrating an attenuation of a radiation amount due to a heel effect of the radiation source.

### Supplementary Note 40

The radiation detection system according to any one of Supplementary Notes 37 to 39, in which a detector included in the radiation detection apparatus includes a plurality of dose detection pixels arrayed to form a plurality of rows and a plurality of columns, the radiation detection system further includes a unit configured to execute communication processing to stop irradiation of radiation from the radiation source in a case where a detection status of the detector satisfies a predetermined condition, and the correction processing includes processing of correcting the predetermined condition.

### Supplementary Note 41

The radiation detection system according to Supplementary Note 40, in which the predetermined condition is a condition that a cumulative value of values obtained by performing correction processing on a dose value obtained from the detector based on the information about the orientation angle and the information about the position at the predetermined timing satisfies a predetermined value.

### Supplementary Note 42

The radiation detection system according to Supplementary Note 40 or 41, in which the correction processing is processing of correcting at least one parameter forming the predetermined condition, and the at least one parameter indicates any one of a cumulative dose threshold for determining whether to stop irradiation of the radiation, a cumulative dose determination method for determining whether to stop irradiation of the radiation, a radiation detection region where a radiation dose is to be monitored, a cumulative dose in the radiation detection region to be monitored, and an average value of cumulative doses in the radiation detection region to be monitored.

### Supplementary Note 43

The radiation detection system according to any one of Supplementary Notes 37 to 42, in which the detector included in the radiation detection apparatus includes a plurality of image capturing pixels for outputting a radiological image corresponding to radiation, and the correction processing includes processing of correcting the radiological image obtained from the plurality of image capturing pixels based on the information about the orientation angle and the information about the position at the predetermined timing.

### Supplementary Note 44

A control apparatus to be used in a radiation detection system including a radiation source configured to irradiate radiation, a radiation detection apparatus configured to detect the radiation, and an optical image capturing unit configured to obtain an optical image by capturing an image of the radiation detection apparatus, the control apparatus including:
a first obtaining unit configured to obtain information about an orientation angle change and information about a position change output from a sensor unit included in the radiation detection apparatus;
a second obtaining unit configured to obtain information about a reference orientation angle and information about a reference position of the radiation detection apparatus based on the optical image; and
a third obtaining unit configured to obtain information about an orientation angle and information about a position of the radiation detection apparatus at a predetermined timing based on the information about the reference orientation angle, the information about the reference position, the information about the orientation angle change, and the information about the position change.

### Supplementary Note 45

A control method for a control apparatus to be used in a radiation detection apparatus including a radiation source configured to irradiate radiation, a radiation detection apparatus configured to detect the radiation, and an optical image capturing unit configured to obtain an optical image by capturing an image of the radiation detection apparatus, the control method including:
a first obtaining step of obtaining information about an orientation angle change and information about a position change output from a sensor unit included in the radiation detection apparatus;
a second obtaining step of obtaining information about a reference orientation angle and information about a reference position of the radiation detection apparatus based on the optical image; and
a third obtaining step of obtaining information about an orientation angle and information about a position of the radiation detection apparatus at a predetermined timing based on the information about the reference orientation angle, the information about the reference position, the information about the orientation angle change, and the information about the position change.

### Supplementary Note 46

A program for causing a computer to execute the control method according to Supplementary Note 45.

### Supplementary Note 47

A radiation detection system that causes a radiation detection apparatus to detect radiation irradiated from a radiation source, the radiation detection system including:
a detector configured to detect a radiation dose;
a unit configured to execute communication processing to stop irradiation of radiation from the radiation source in a case where a detection status of the detector satisfies a predetermined condition;
a unit configured to obtain first orientation information corresponding to an orientation of the detector and second orientation information corresponding to an orientation of the radiation source; and
a unit configured to correct the predetermined condition based on at least the first orientation information and the second orientation information.

### Supplementary Note 48

A radiation detection apparatus configured to detect radiation irradiated from a radiation source, the radiation detection apparatus including:
a detector configured to detect a radiation dose;
a unit configured to execute communication processing to stop irradiation of radiation from the radiation source in a case where a detection status of the detector satisfies a predetermined condition;
a unit configured to obtain first orientation information corresponding to an orientation of the detector and second orientation information corresponding to an orientation of the radiation source; and
a unit configured to correct the predetermined condition based on at least the first orientation information and the second orientation information.

The present invention is not limited to the above-described exemplary embodiments and can be modified or altered in various ways without departing from the spirit and scope of the present invention. Accordingly, the following claims are attached to publicize the scope of the present invention.

According to the present disclosure, it is possible to provide a technique for accurately deriving an orientation of a radiation imaging apparatus while reducing the time and effort for a user, such as an engineer, to perform an operation.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD) TM), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A radiation detection system including a radiation source configured to irradiate radiation and a radiation detection apparatus configured to detect the radiation, the radiation detection system comprising:
an optical image capturing unit configured to obtain an optical image by capturing an image of the radiation detection apparatus; and
one or more controllers configured to:
obtain information about an orientation angle change and/or information about a position change output from a sensor unit included in the radiation detection apparatus;
obtain information about a reference orientation angle and/or information about a reference position of the radiation detection apparatus based on the optical image; and
obtain information about an orientation angle at a predetermined timing based on the information about the reference orientation angle and the information about the orientation angle change and/or information about a position at a predetermined timing based on the information about the reference position and the information about the position change.

2. The radiation detection system according to claim 1, wherein the reference orientation angle and the reference position respectively correspond to an angle and a position relative to the radiation source.

3. The radiation detection system according to claim 1, wherein the sensor unit includes at least an acceleration sensor and a gyroscope sensor.

4. The radiation detection system according to claim 1, further comprising a control apparatus including the one or more controllers.

5. The radiation detection system according to claim 4,
wherein the radiation imaging apparatus includes a plurality of markers on a surface of the radiation imaging apparatus, and
wherein, in a case where the optical image includes two or more of the plurality of markers, the control apparatus derives the reference orientation angle and the reference position based on the optical image.

6. The radiation detection system according to claim 5, wherein each of the plurality of markers is a two-dimensional code.

7. The radiation detection system according to claim 4,
wherein the one or more controllers detect a stationary state of the radiation detection apparatus based on output from the sensor unit or a plurality of optical images, and
wherein the radiation detection system obtains information about an orientation angle and information about a position of the radiation detection apparatus at a predetermined timing based on the output from the sensor unit and the optical image in a state where the radiation detection apparatus is in the stationary state.

8. The radiation detection system according to claim 1, wherein the one or more controllers execute correction processing based on the information about the orientation angle and the information about the position at the predetermined timing.

9. The radiation detection system according to claim 8, wherein the correction processing includes processing of calibrating an attenuation of a radiation amount due to a grid.

10. The radiation detection system according to claim 8, wherein the correction processing includes processing of calibrating an attenuation of a radiation amount due to a heel effect of the radiation source.

11. The radiation detection system according to claim 8,
wherein a detector included in the radiation detection apparatus includes a plurality of dose detection pixels arrayed to form a plurality of rows and a plurality of columns,
wherein the one or more controllers execute communication processing for stopping irradiation of radiation from the radiation source in a case where a detection status of the detector satisfies a predetermined condition, and
wherein the correction processing includes processing of correcting the predetermined condition.

12. The radiation detection system according to claim 11, wherein the predetermined condition is a condition that a cumulative value of values obtained by performing correction processing on a dose value obtained from the detector based on the information about the orientation angle and the information about the position at the predetermined timing satisfies a predetermined value.

13. The radiation detection system according to claim 11,
wherein the correction processing is processing of correcting at least one parameter forming the predetermined condition, and
wherein the at least one parameter indicates any one of a cumulative dose threshold for determining whether to stop irradiation of the radiation, a cumulative dose determination method for determining whether to stop irradiation of the radiation, a radiation detection region where a radiation dose is to be monitored, a cumulative dose in the radiation detection region to be monitored, and an average value of cumulative doses in the radiation detection region to be monitored.

14. The radiation detection system according to claim 8,
wherein the detector included in the radiation detection apparatus includes a plurality of image capturing pixels for outputting a radiological image corresponding to radiation, and
wherein the correction processing includes processing of correcting the radiological image obtained from the plurality of image capturing pixels based on the information about the orientation angle and the information about the position at the predetermined timing.

15. A control apparatus for a radiation detection system including a radiation source configured to irradiate radiation, a radiation detection apparatus configured to detect the radiation, and an optical image capturing unit configured to obtain an optical image by capturing an image of the radiation detection apparatus, the control apparatus comprising:
one or more controllers configured to:
obtain information about an orientation angle change and/or information about a position change output from a sensor unit included in the radiation detection apparatus;
obtain information about a reference orientation angle and/or information about a reference position of the radiation detection apparatus based on the optical image; and
obtain information about an orientation angle at a predetermined timing based on the information about the reference orientation angle and the information about the orientation angle change and/or information about a position at a predetermined timing based on the information about the reference position and the information about the position change.

16. A control method for a control apparatus to be used in a radiation detection system including a radiation source configured to irradiate radiation, a radiation detection apparatus configured to detect the radiation, and an optical image capturing unit configured to obtain an optical image by capturing an image of the radiation detection apparatus, the control method comprising:
obtaining information about an orientation angle change and/or information about a position change output from a sensor unit included in the radiation detection apparatus;
obtaining information about a reference orientation angle and/or information about a reference position of the radiation detection apparatus based on the optical image; and
obtaining information about an orientation angle at a predetermined timing based on the information about the reference orientation angle and the information about the orientation angle change and/or information about a position at a predetermined timing based on the information about the reference position and the information about the position change.

17. A computer-readable storage medium storing a program for causing a computer to execute the control method according to claim 16.

18. A radiation detection system that causes a radiation detection apparatus to detect radiation irradiated from a radiation source, the radiation detection system comprising:
a detector configured to detect a radiation dose; and
one or more controllers configured to:
execute communication processing to stop irradiation of radiation from the radiation source in a case where a detection status of the detector satisfies a predetermined condition;
obtain first orientation information corresponding to an orientation of the detector and second orientation information corresponding to an orientation of the radiation source; and
correct the predetermined condition based on at least the first orientation information and the second orientation information.

19. A radiation detection apparatus configured to detect radiation irradiated from a radiation source, the radiation detection apparatus comprising:
a detector configured to detect a radiation dose; and
one or more controllers configured to:
execute communication processing to stop irradiation of radiation from the radiation source in a case where a detection status of the detector satisfies a predetermined condition;
obtain first orientation information corresponding to an orientation of the detector and second orientation information corresponding to an orientation of the radiation source; and
correct the predetermined condition based on at least the first orientation information and the second orientation information.
